(19) United States
(12) Patent Application Publication
(10) Pub. No.: US 2021/0405061 A1
(43) Pub. Date: Dec. 30, 2021

(54) METHOD FOR MEASURING AB PEPTIDE

(71) Applicant: SYSMEX CORPORATION, Kobe-shi (JP)

(72) Inventors: Takuya IINO, Kobe-shi (JP); Shunsuke WATANABE, Kobe-shi (JP); Kazuto YAMASHITA, Kobe-shi (JP); Kouzou SUTO, Kobe-shi (JP)

(73) Assignee: SYSMEX CORPORATION, Kobe-shi (JP)

(21) Appl. No.: 17/101,303

(22) Filed: Nov. 23, 2020

(30) Foreign Application Priority Data
Jun. 25, 2020 (JP) 2020-109829

(51) Int. Cl. G01N 33/68

(52) U.S. Cl. CPC G01N 33/6848; G01N 33/6857

(57) ABSTRACT

Disclosed is a method for measuring an Aβ peptide in a blood sample in vitro, comprising: measuring the Aβ peptide by an immunoassay using an antibody set comprising a capture antibody and a detection antibody that specifically bind to the Aβ peptide, wherein the capture antibody is an antibody that binds to an epitope comprising an N-terminal residue of the Aβ peptide, the detection antibody is an antibody that binds to an epitope different from the epitope to which the capture antibody binds, and the Aβ peptide is at least one selected from the group consisting of Aβ40 or Aβ42.

### CROSS REFERENCE TO RELATED APPLICATIONS

This application claims priority from prior Japanese Patent Application No. 2020-109829, filed on Jun. 25, 2020, entitled “Antibody set for measuring Aβ peptide, method for measuring Aβ peptide and reagent kit”, the entire contents of which are incorporated herein by reference.

### FIELD OF THE INVENTION

The present invention relates to a method for measuring an Aβ peptide.

### BACKGROUND

An Aβ peptide in a biological sample collected from a subject is known to be a biomarker for Alzheimer's disease. Since cerebrospinal fluid (CSF) contains a relatively large amount of Aβ peptide among biological samples, a method for quantitatively measuring an Aβ peptide in CSF has been established. For example, Leinenbach A. et al., Mass Spectrometry-Based Candidate Reference Measurement Procedure for Quantification of Amyloid-β in Cerebrospinal Fluid. Clinical Chemistry 60: 7 987-994 (2014) describes that an Aβ peptide in CSF is measured by liquid chromatography-mass spectrometry (LC-MS).

Since collection of CSF is invasive, burden on a subject is large. Therefore, recently, a method for measuring an Aβ peptide using blood, which has a low collection burden, as a biological sample has been developed. However, since the amount of Aβ peptide contained in blood is less than that in CSF, a method for accurately measuring an Aβ peptide has been required. An object of the present invention is to provide a means for enabling accurate measurement of Aβ peptide in blood.

### SUMMARY OF THE INVENTION

The scope of the present invention is defined solely by the appended claims, and is not affected to any degree by the statements within this summary.

The present invention provides a method for measuring an Aβ peptide using an antibody set comprising a capture antibody and a detection antibody that specifically bind to the Aβ peptide. The capture antibody is an antibody that binds to an epitope comprising an N-terminal residue of the Aβ peptide, and the detection antibody is an antibody that binds to an epitope different from the epitope to which the capture antibody binds, and the Aβ peptide is at least one selected from the group consisting of Aβ40 or Aβ42.

The present invention provides a method for measuring an Aβ peptide in a blood sample in vitro, comprising: forming on a solid phase a complex comprising a capture antibody, the Aβ peptide and a detection antibody, the detection antibody being labeled with a labeling substance; and detecting the complex based on the labeling substance in the complex whereby the Aβ peptide is measured, wherein the capture antibody is an antibody that binds to an epitope comprising an N-terminal residue of the Aβ peptide, and the detection antibody is an antibody that binds to an epitope different from the epitope to which the capture antibody binds, and the Aβ peptide is at least one selected from the group consisting of Aβ40 or Aβ42.

The present invention provides a method for measuring an A3 peptide in a blood sample in vitro, comprising: measuring an Aβ40 by an immunoassay using: a capture antibody which is at least one selected from the group consisting of an 82E1 antibody and a 2H4 antibody; and a detection antibody which is a 1A10 antibody, and measuring an Aβ42 by an immunoassay using: a capture antibody which is at least one selected from the group consisting of an 82E1 antibody and a 2H4 antibody; and a detection antibody which is an H31L21 antibody.

### BRIEF DESCRIPTION OF THE DRAWINGS

FIG. 1A is a view showing an example of an appearance of a reagent kit according to this embodiment;

FIG. 1B is a view showing an example of an appearance of a reagent kit according to this embodiment;

FIG. 1C is a view showing an example of an appearance of a reagent kit according to this embodiment;

FIG. 1D is a view showing an example of an appearance of a reagent kit according to this embodiment;

FIG. 2 is a diagram showing results of measurement of Aβ peptide using LC-MS/MS;

FIG. 3A is a diagram showing concentrations of Aβ40 peptide in plasma of each specimen;

FIG. 3B is a diagram showing concentrations of Aβ42 peptide in plasma of each specimen;

FIG. 4A is a diagram showing a calibration curve prepared based on measured values detected using an Aβ40 peptide with a known concentration;

FIG. 4B is a diagram showing a calibration curve prepared based on measured values detected using an Aβ42 peptide with a known concentration;

FIG. 5 is a diagram showing difference in elution efficiency of Aβ peptide due to a difference in composition of releasing agent;

FIG. 6A is a diagram showing difference in elution efficiency of Aβ40 peptide due to a difference in organic solvent of releasing agent;

FIG. 6B is a diagram showing difference in elution efficiency of Aβ42 peptide due to a difference in organic solvent of releasing agent;

FIG. 7 is a diagram showing a difference in amount of carryover due to a difference in composition of releasing agent;

FIG. 8A is a diagram showing an amount of Aβ40 peptide when a 190 pg/ml Aβ40 peptide solution is eluted with a basic solution;

FIG. 8B is a diagram showing an amount of Aβ40 peptide when a 190 pg/ml Aβ40 peptide solution is eluted with an acidic solution;

FIG. 8C is a diagram showing an amount of Aβ42 peptide when a 103 pg/ml Aβ42 peptide solution is eluted with a basic solution;

FIG. 8D is a diagram showing an amount of Aβ42 peptide when a 103 pg/ml Aβ42 peptide solution is eluted with an acidic solution;

FIG. 9A is a diagram showing an amount of Aβ40 peptide when a 50 pg/ml Aβ40 peptide solution is eluted with a basic solution;

FIG. 9B is a diagram showing an amount of Aβ40 peptide when a 50 pg/ml Aβ40 peptide solution is eluted with an acidic solution;

FIG. 9C is a diagram showing an amount of Aβ42 peptide when a 26 pg/ml Aβ42 peptide solution is eluted with a basic solution;

FIG. 9D is a diagram showing an amount of Aβ42 peptide when a 26 pg/ml Aβ42 peptide solution is eluted with an acidic solution;

FIG. 10A is a diagram showing a result of obtaining correlation coefficient by plotting measured values of Aβ40 peptide of a plasma sample measured by HISCL (registered trademark)-5000 against measured values of Aβ40 peptide of the plasma sample measured by immunoprecipitation and mass spectrometry;

FIG. 10B is a diagram showing a result of obtaining correlation coefficient by plotting measured values of Aβ40 peptide of a peptide spike sample measured by HISCL (registered trademark)-5000 against measured values of Aβ40 peptide of the peptide spike sample measured by immunoprecipitation and mass spectrometry;

FIG. 10C is a diagram showing a result of obtaining correlation coefficient by plotting measured values of Aβ40 peptide of a plasma sample and a peptide spike sample measured by HISCL (registered trademark)-5000 against measured values of Aβ40 peptide of the plasma sample and the peptide spike sample measured by immunoprecipitation and mass spectrometry;

FIG. 11A is a diagram showing a result of obtaining correlation coefficient by plotting measured values of Aβ42 peptide of a plasma sample measured by HISCL (registered trademark)-5000 against measured values of Aβ42 peptide of the plasma sample measured by immunoprecipitation and mass spectrometry;

FIG. 11B is a diagram showing a result of obtaining correlation coefficient by plotting measured values of Aβ42 peptide of a peptide spike sample measured by HISCL (registered trademark)-5000 against measured values of Aβ42 peptide of the peptide spike sample measured by immunoprecipitation and mass spectrometry;

FIG. 11C is a diagram showing a result of obtaining correlation coefficient by plotting measured values of Aβ42 peptide of a plasma sample and a peptide spike sample measured by HISCL (registered trademark)-5000 against measured values of Aβ42 peptide of the plasma sample and the peptide spike sample measured by immunoprecipitation and mass spectrometry; and

FIG. 12 is a diagram showing a result of obtaining correlation coefficient by plotting measured values of Aβ42 peptide of a plasma sample measured by HISCL (registered trademark)-5000 using 6E10 antibody as a capture antibody against measured values of Aβ42 peptide of the plasma sample measured by immunoprecipitation and mass spectrometry.

### DETAILED DESCRIPTION OF THE PREFERRED EMBODIMENTS

One embodiment is an antibody set for measuring an Aβ peptide in a blood sample by immunoassay. The antibody set refers to a combination of a plurality of antibodies including at least a capture antibody and a detection antibody used in immunoassay. The type of immunoassay is not particularly limited. For example, the type of immunoassay can be selected as appropriate from known immunoassay methods such as enzyme-linked immunosorbent assay (ELISA), immunoprecipitation/Western blotting, and immune complex transfer method (see Japanese Laid-Open Patent Publication No. H1-254868). Among them, the ELISA is preferred. The type of the ELISA may be any of a sandwich method, a competitive method, a direct method, an indirect method and the like, and the sandwich method is preferred. The immunoassay using the antibody set of this embodiment may be performed by a commercially available immunoassay apparatus such as HISCL (registered trademark) series (Sysmex Corporation).

As used herein, the term “blood sample” includes blood samples containing an Aβ peptide and blood samples suspected of containing an Aβ peptide. Examples of the blood samples include blood (whole blood), plasma, serum, and the like. Of these, plasma and serum are preferred. The blood sample may be diluted with an appropriate aqueous medium as necessary. The aqueous medium is not particularly limited as long as it does not interfere with the measurement described later. Examples of the aqueous medium include water, physiological saline, a buffer solution, and the like. The buffer solution is a buffer solution having a buffering effect at a pH near neutrality (for example, a pH of 6 or more and 8 or less). Examples of the buffer solution include Good buffers such as HEPES, MES, and PIPES, tris buffered saline (TBS), phosphate buffered saline (PBS), and the like.

An origin of the blood sample is not particularly limited. For example, the blood sample may be blood collected from a subject and plasma or serum prepared from the blood. Commercially available pool plasma, healthy person plasma or the like may be used. A labeled Aβ peptide as an internal standard substance may be added to the blood sample as necessary. The subject is not particularly limited. Examples of the subject include a healthy person, a person having abnormality in cognitive function, and a person suspected of having the abnormality. Examples of cognitive dysfunction include mild cognitive impairment (MCI), Alzheimer's disease, and the like.

Aβ peptide is a polypeptide produced by treating amyloid R precursor protein (APP) with D-secretase and γ-secretase. Unless otherwise specified, Aβ peptides include polypeptides of any length, but are usually polypeptides consisting of 39 to 43 amino acids. As the Aβ peptide, Aβ40 (DAEFRHDSGYEVHHQKLVFFAEDVGSNKGAIIGLMVGGVV: SEQ ID NO: 1) consisting of 40 amino acid residues and Aβ42 (DAEFRHDSGYEVHHQKLVFFAEDVGSNKGAIIGLMVGGVVIA: SEQ ID NO: 2) consisting of 42 amino acid residues are preferable.

The Aβ peptide may be in the form of a monomer or a multimer. A multimer, also called a polymer, is formed by physically or chemically polymerizing or aggregating a plurality of monomeric Aβ peptides. The multimer may contain a plurality of monomeric Aβ peptides, and may contain other molecules. In the multimer, the monomeric Aβ peptides do not need to be tightly bound to each other by covalent bonds or the like. Multimers also include aggregates in which a plurality of monomeric Aβ peptides are aggregated by looser binding. Examples of the multimer of Aβ peptide include Aβ oligomers and the like.

As used herein, the term “antibody” includes full-length antibodies and fragments thereof. Examples of the antibody fragments include Fab, Fab′, F(ab′)2, Fd, Fd′, Fv, light chain, heavy chain variable region (VHH) of heavy chain antibody, reduced IgG (rIgG), one chain antibodies (scFv), and the like. The antibody that specifically binds to an Aβ peptide may be either a monoclonal antibody or a polyclonal antibody, but is preferably a monoclonal antibody.

The capture antibody is an antibody that specifically binds to a test substance and is immobilized on a solid phase. By binding the test substance and the capture antibody, the test substance is immobilized on the solid phase. The detection antibody is an antibody that specifically binds to the test substance. The detection antibody is preferably labeled with a labeling substance. The labeling substance is as described later. The detection antibody is usually not immobilized on the solid phase, but the labeling substance itself may be a solid phase carrier such as a particle. The antibody set of this embodiment contains a capture antibody and a detection antibody that specifically bind to the Aβ peptide. A capture antibody that specifically binds to an Aβ peptide is an antibody that binds to an epitope including an N-terminal residue of the Aβ peptide. A detection antibody that specifically binds to an Aβ peptide is an antibody that binds to an epitope different from the epitope to which the capture antibody binds. In the antibody set of this embodiment, the Aβ peptide is at least one of Aβ40 or Aβ42.

In this embodiment, the epitope including the N-terminal residue of the Aβ peptide to which the capture antibody binds refers to a region including the N-terminal residue of the Aβ peptide and consisting of a part of amino acid sequence of the Aβ peptide. The preferred epitope of the capture antibody is contained in a region containing the N-terminal residue of the Aβ peptide and consisting of 1st to 16th amino acid residues counting from the N-terminus of the Aβ peptide. Examples of the epitopes include regions consisting of 1st to 5th, 1st to 6th, 1st to 7th, 1st to 8th, 1st to 9th, 1st to 10th, 1st to 11th, 1st to 12th, 1st to 13th, 1st to 14th, 1st to 15th or 1st to 16th amino acid residues counting from the N-terminus of the Aβ peptide. Examples of antibodies that bind to the epitopes include an antibody of clone 82E1 (called 82E1 antibody) that recognizes 1st to 16th regions counting from the N-terminal amino acid residue of the Aβ peptide as an epitope, and an antibody of clone 2H4 (called 2H4 antibody) that recognizes 1st to 8th regions counting from the N-terminus of the Aβ peptide as an epitope. The 82E1 antibody is particularly preferable among them. These monoclonal antibodies are commercially available.

In this embodiment, the epitope to which the detection antibody binds is different from the epitope to which the capture antibody binds. In other words, the capture antibody and the detection antibody do not substantially compete for binding to the antigen Aβ peptide. The epitope of the detection antibody preferably contains a C-terminal residue of the Aβ peptide. The epitope including the C-terminal residue of the Aβ peptide to which the detection antibody binds refers to a region containing the C-terminal residue of the Aβ peptide and consisting of a part of amino acid sequence of the Aβ peptide. The preferred epitope of the detection antibody is contained in a region containing the C-terminal residue of the Aβ peptide and consisting of 35th to 40th amino acid residues counting from an N-terminus of Aβ40 or 36th to 42nd amino acid residue counting from an N-terminus of Aβ42. Examples of the epitopes include a region consisting of 35th to 40th amino acid residues counting from the N-terminus of Aβ40 and a region consisting of 36th to 42th amino acid residues counting from the N-terminus of Aβ42. Examples of antibodies that bind to the epitope including the C-terminal residue of the Aβ peptide include an antibody of clone H31L21 (called H31L21 antibody) that recognizes 36th to 42nd regions counting from the N-terminus of Aβ42 as an epitope, and an antibody of clone 1A10 (called 1A10 antibody) that recognizes 35th to 40th regions counting from the N-terminus of Aβ40 as an epitope. These monoclonal antibodies are commercially available.

PCT International Application Publication No. 2007/022015 A describes a measurement method using an antibody set of a combination different from that of the antibody set of this embodiment. In the method described in this document, 1A10 antibody is used as the capture antibody and 82E1 antibody is used as the detection antibody. The document states that use of 82E1 antibody as the capture antibody reduces sensitivity of Aβ detection. However, in this embodiment, highly sensitive detection of Aβ peptide is possible by using an antibody that binds to the epitope including the N-terminal residue of the Aβ peptide like 82E1 antibody as the capture antibody in immunoassay.

The capture antibody and the detection antibody included in the antibody set may be either a monoclonal antibody or a polyclonal antibody, respectively. The capture antibody and the detection antibody are preferably monoclonal antibodies. In a preferred embodiment, both the capture antibody and the detection antibody are monoclonal antibodies.

In this embodiment, the capture antibody is preferably previously immobilized on a solid phase. The solid phase may be any insoluble carrier capable of immobilizing an antibody. The mode of immobilization of the antibody on the solid phase is not particularly limited. For example, the antibody and the solid phase may be bound directly, or the antibody and the solid phase may be indirectly bound via another substance. Examples of the direct binding include physical adsorption and the like. Examples of the indirect binding include immobilizing a molecule that specifically binds to an antibody on a solid phase, and immobilizing the antibody on the solid phase through binding between the molecule and the antibody. Examples of the molecule that specifically binds to the antibody include protein A or G, an antibody (a secondary antibody) that specifically recognizes an antibody, and the like. A combination of substances interposed between the antibody and the solid phase can be used to immobilize the antibody on the solid phase. Examples of the combination of substances include combinations of any of biotins and any of avidins, a hapten and an anti-hapten antibody and the like. The biotins include biotin and biotin analogs such as desthiobiotin and oxybiotin. The avidins include avidin and analogs of avidins such as streptavidin and tamavidin (registered trademark). Examples of the combination of a hapten and an anti-hapten antibody include a combination of a compound having a 2,4-dinitrophenyl (DNP) group and an anti-DNP antibody. For example, by using an antibody previously modified with a biotin (or a compound having a DNP group) and a solid phase to which an avidin (or anti-DNP antibody) is previously bound, the antibody can be immobilized on the solid phase through binding between the biotin and the avidin (or binding between the DNP group and the anti-DNP antibody).

The material of the solid phase is not particularly limited. For example, the material can be selected from organic polymer compounds, inorganic compounds, biopolymers, and the like. Examples of the organic polymer compounds include latex, polystyrene, polypropylene, styrene-methacrylic acid copolymer, styrene-glycidyl (meth)acrylate copolymer, styrene-styrene sulfonate copolymer, methacrylic acid polymer, acrylic acid polymer, acrylonitrile butadiene styrene copolymer, vinyl chloride-acrylate copolymer, polyvinyl acetate acrylate, and the like. Examples of the inorganic compounds include magnetic bodies (iron oxide, chromium oxide, cobalt, ferrite, etc.), silica, alumina, glass, and the like. Examples of the biopolymer include insoluble agarose, insoluble dextran, gelatin, cellulose, and the like. Two or more of these may be used in combination. The shape of the solid phase is not particularly limited. Examples of the shape of the solid phase include a particle, a microplate, a microtube, a test tube, and the like. Among them, particles are preferable, and magnetic particles are particularly preferable.

The detection antibody is preferably labeled with a labeling substance. The labeling substance is not particularly limited. For example, the labeling substance may be a substance which itself generates a signal (hereinafter also referred to as “signal generating substance”) or a substance which catalyzes the reaction of other substances to generate a signal. Examples of the signal generating substance include fluorescent substances, radioactive isotopes, and the like. Examples of the fluorescent substances include fluorescent dyes such as fluorescein isothiocyanate (FITC), rhodamine and Alexa Fluor (registered trademark), fluorescent proteins such as GFP, and the like. Examples of the radioactive isotopes include ¹²⁵I, ¹⁴C, ³²P, and the like. Examples of the substance that catalyzes the reaction of other substances to generate a detectable signal include enzymes. The labeling substance is more preferably an enzyme. Examples of the enzyme include alkaline phosphatase, peroxidase, β-galactosidase, glucosidase, polyphenol oxidase, tyrosinase, acid phosphatase, luciferase, and the like. Among them, alkaline phosphatase is particularly preferable.

Methods for detecting a signal themselves are known in the art. In this embodiment, a measurement method according to the type of signal derived from the labeling substance may be appropriately selected. For example, when the labeling substance is an enzyme, signals such as light and color generated by reacting a substrate for the enzyme can be measured by using a known apparatus such as a spectrophotometer.

The substrate of the enzyme can be appropriately selected from known substrates according to the type of the enzyme. For example, when alkaline phosphatase is used as the enzyme, examples of the substrate include chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate) and CSPD (registered trademark) (disodium 3-(4-methoxyspiro[1,2-dioxetane-3,2-(5′-chloro)tricyclo[3.3.1.13,7]decan]-4-yl)phenyl phosphate), and chromogenic substrates such as 5-bromo-4-chloro-3-indolyl phosphate (BCIP), disodium 5-bromo-6-chloro-indolyl phosphate and p-nitrophenyl phosphate. When peroxidase is used as the enzyme, examples of the substrate include chemiluminescent substrates such as luminol and derivatives thereof, and chromogenic substrates such as 2,2′-azinobis(3-ethylbenzothiazoline-6-ammonium sulfonate) (ABTS), 1,2-phenylenediamine (OPD) and 3,3′,5,5′-tetramethylbenzidine (TMB).

When the labeling substance is a radioactive isotope, radiation as a signal can be measured using a known apparatus such as a scintillation counter. When the labeling substance is a fluorescent substance, fluorescence as a signal can be measured using a known apparatus such as a fluorescence microplate reader. The excitation wavelength and the fluorescence wavelength can be appropriately determined according to the type of fluorescent substance used.

As an example of the immunoassay, a case where an Aβ peptide in a blood sample is measured by sandwich ELISA using the antibody set of this embodiment will be described below. First, the blood sample and the capture antibody are mixed to form a complex of the Aβ peptide and the capture antibody. Then, the complex is formed on the solid phase by contacting a solution containing the complex with a solid phase on which the capture antibody can be immobilized. Alternatively, a solid phase previously immobilized with the capture antibody may be used. That is, the solid phase on which the capture antibody is immobilized is contacted with the blood sample to form a complex of the Aβ peptide and the capture antibody in the blood sample on the solid phase. Subsequently, the complex formed on the solid phase is contacted with a detection antibody to form a sandwich complex of the Aβ peptide, the capture antibody and the detection antibody on the solid phase.

Then, the complex formed on the solid phase is detected by a method known in the art, whereby a measured value of the Aβ peptide in the blood sample can be acquired. For example, when an antibody labeled with a labeling substance is used as a detection antibody, the measured value of the Aβ peptide in the blood sample can be acquired by detecting a signal generated by the labeling substance. Alternatively, also when a labeled secondary antibody against the detection antibody is used, the measured value of the Aβ peptide can be acquired in the same manner. The labeling substance is as described above.

As used herein, the phrase “detecting a signal” includes qualitatively detecting the presence or absence of a signal, quantifying a signal intensity, and semi-quantitatively detecting the intensity of a signal. Semi-quantitative detection means to show the intensity of the signal in stages like “no signal generated”, “weak”, “medium”, “strong”, and the like. In this embodiment, it is preferable to detect the intensity of the signal quantitatively.

The detection result of the signal can be used as the measurement result of the Aβ peptide in the blood sample. For example, when quantitatively detecting the intensity of a signal, a measured value of the signal intensity itself or a value acquired from the measured value can be used as the measurement result of the Aβ peptide. Examples of the value acquired from the measured value of the signal intensity include a value acquired by subtracting the measured value of a negative control sample or the background value from the measured value, and the like. The negative control can be appropriately selected, and is, for example, a solution containing no Aβ peptide (for example, a buffer solution).

In this embodiment, the measured value of the signal intensity may be applied to a calibration curve to determine the amount or concentration value of the Aβ peptide. The calibration curve can be prepared from measured values obtained by measuring a calibrator containing the Aβ peptide at a known concentration by the immunoassay of this embodiment in the same manner as the blood sample. Specifically, the measured values of Aβ peptide acquired from a plurality of calibrators are plotted on an XY plane in which the concentration of Aβ peptide in the calibrator is taken on an X-axis and the measured values (for example, signal intensities) are taken on a Y-axis to obtain a straight line or a curve by a known method such as a least squares method, whereby a calibration curve can be prepared. The calibrator containing the Aβ peptide at a known concentration can be prepared, for example, by adding synthetic Aβ peptide at an arbitrary concentration to a buffer solution containing no Aβ peptide.

In the immunoassay using the antibody set of this embodiment, B/F separation for removing an unreacted free component not forming a complex may be performed between the formation of the complex and the detection. B/F separation will be described later.

The immunoassay using the antibody set of this embodiment may be performed by a commercially available fully automated immunoassay system. A fully automated immunoassay system is a system for automatically performing preparation of measurement sample and its immunoassay, when a user sets a biological sample such as a blood sample and inputs an instruction to start measurement, and outputting a measurement result of a test substance. Examples of the fully automated immunoassay system include HISCL (registered trademark) series (Sysmex Corporation) or HI-1000 (Sysmex Corporation) such as HISCL (registered trademark)-5000 and HISCL (registered trademark)-800.

On the other hand, an Aβ peptide can be measured by a method for measuring an Aβ peptide captured by an antibody by mass spectrometry (hereinafter, also referred to as “measurement method”). This measurement method is characterized in that the Aβ peptide is released from the complex of the Aβ peptide and the antibody with a basic solution containing an organic solvent, and the released Aβ peptide is measured by mass spectrometry. In the present specification, the “basic solution” means a solution whose pH is basic.

Conventionally, when a test substance is released from the test substance captured by the antibody (that is, the complex of the antibody and the test substance), an acidic solution is used. This is because an acidic solution generally has an excellent action of dissociating a binding between a test substance and an antibody. For example, Japanese Laid-Open Patent Publication No. 2018-194374 describes that an Aβ peptide is released from the complex by an acidic aqueous solution with a pH of 1 to 4, which may contain an organic solvent. However, as shown in the examples described later, the present inventors found that an acidic solution containing a released Aβ peptide is not suitable for mass spectrometry in combination with liquid chromatography. Specifically, when the acidic solution containing the released Aβ peptide was used as it was for liquid chromatography, a phenomenon in which a part of the charged Aβ peptides remained in a flow path and column of a liquid chromatography apparatus and the remaining Aβ peptide was carried over to measurement of next sample (called carryover) occurred. When carryover occurs, accurate measurement of Aβ peptide cannot be performed. In order to reduce carryover, it is conceivable to release the Aβ peptide in an acidic solution and then exchange the solvent with a basic solution. However, even if the carryover can be reduced by this method, it may be difficult to measure with high sensitivity because the Aβ peptide is lost due to solvent exchange. On the other hand, in the measurement method of this embodiment using a basic solution containing an organic solvent, the amount of Aβ peptide carried over is significantly reduced. Therefore, even if the basic solution containing the released Aβ peptide is used as it is for liquid chromatography, the Aβ peptide can be accurately measured and no solvent exchange is required.

In the above measurement method, first, an antibody that specifically binds to an Aβ peptide is mixed with a blood sample to form a complex of the Aβ peptide and the antibody. A complex of an Aβ peptide and an antibody can be formed by mixing a blood sample with an antibody that specifically binds to the Aβ peptide.

In the above measurement method, the antibody is preferably a monoclonal antibody capable of specifically binding to an Aβ peptide. Examples of the antibody include 82E1 antibody, an antibody of clone 6E10 (called 6E10 antibody) that recognizes 3rd to 8th regions counting from the N-terminal amino acid residue of the Aβ peptide as an epitope, an antibody of clone WO-2 (called WO-2 antibody) that recognizes 4th to 10th regions counting from the N-terminal amino acid residue of the Aβ peptide as an epitope, 2H4 antibody, H31L21 antibody, an antibody of clone G2-11 (called G2-11 antibody) that recognizes 33th to 42nd regions counting from the N-terminal amino acid residue of Aβ42 as an epitope, an antibody of clone 16C11 (called 16C11 antibody) that recognizes 33th to 42nd regions counting from the N-terminal amino acid residue of Aβ42 as an epitope, an antibody of clone 21F12 (called 21F12 antibody) that recognizes 34th to 42nd regions counting from the N-terminal amino acid residue of Aβ42 as an epitope, 1A10 antibody, and the like. These monoclonal antibodies are commercially available.

In the above measurement method, it is preferable to use an antibody that binds to one or both of Aβ40 and Aβ42 as an antibody that specifically binds to the Aβ peptide. Examples of the antibody that specifically binds to Aβ40 include an antibody of clone 1A10. Examples of the antibody that specifically binds to Aβ42 include antibodies of clones H31L21, G2-11, 16C11 and 21F12. Examples of the antibody that binds to both Aβ40 and Aβ42 include antibodies of clones 82E1, 6E10, WO-2 and 2H4. By using these antibodies, Aβ40 and/or Aβ42 can be selectively captured among the Aβ peptides in the blood sample. In this case, at least one of Aβ40 or Aβ42 can be released in releasing of the measurement method of this embodiment, and at least one measured value of Aβ40 or Aβ42 can be acquired in measuring.

In the above measurement method, it is preferable to use an antibody that binds to both Aβ40 and Aβ42. In this case, Aβ40 and Aβ42 can be released in the releasing of the above measurement method, and a measured value of Aβ40 and a measured value of Aβ42 can be acquired in the measuring.

In the above measurement method, it is preferable to form a complex of the Aβ peptide and the antibody on the solid phase in order to selectively acquire the Aβ peptide captured by the antibody. The complex can be formed on the solid phase by contacting a solution containing the complex with a solid phase on which the antibody can be immobilized. Alternatively, an antibody that specifically binds to the Aβ peptide may be previously immobilized on the solid phase and used. By using an antibody immobilized on the solid phase, the complex can be formed on the solid phase. Specifically, the complex is formed on the solid phase by mixing the solid phase on which the antibody that specifically binds to the Aβ peptide is immobilized and the blood sample. Then, the complex can be selectively acquired by separating an unreacted free component and the solid phase and recovering the solid phase. When a particle is used as the solid phase, a complex forming in the above measurement method corresponds to a general immunoprecipitation method.

Subsequently, in the above measurement method, the Aβ peptide is released from the complex with a basic solution containing an organic solvent (hereinafter, also referred to as “releasing agent”). The releasing agent is considered to have an action of dissociating the binding between the antibody and the Aβ peptide. In a preferred embodiment, the solution containing the complex formed on the solid phase is mixed with the releasing agent. As a result, the Aβ peptide is released from the complex, and the released Aβ peptide and the solid phase on which the antibody is immobilized are present in the mixed solution. For example, when the solid phase is a magnetic particle, a solution containing the Aβ peptide can be recovered by separating the solid phase on which the antibody is immobilized and the solution containing the Aβ peptide by centrifugation or magnetic separation.

Conditions for contact between the complex and the releasing agent are not particularly limited. For example, the releasing agent at a temperature of 4° C. or more and 42° C. or less is contacted with the complex and allowed to stand or agitated for 1 minute or more and 10 minutes or less. An amount of the releasing agent used is not particularly limited. For example, the amount can be appropriately determined, for example, from the range of 10 μL or more and 50 μL or less per sample.

The releasing agent can be obtained by mixing a basic solution with an organic solvent, or by mixing water with a basic substance and an organic solvent. The basic solution can be obtained by mixing water and a basic substance. A commercially available basic solution such as aqueous ammonia may be used. Examples of the basic substance include a substance that donates an ammonium ion and the like. Examples of the substance that donates an ammonium ion include ammonia, ammonium carbonate, ammonium bicarbonate, and the like. The basic substance in the releasing agent may be one type or two or more types.

Examples of the organic solvent include acetonitrile, acetone, 1-propanol, 2-propanol, hexane, ethanol, dimethyl sulfoxide, methanol, and the like. The organic solvent in the releasing agent may be one type or two or more types. In this embodiment, the releasing agent preferably contains at least acetonitrile as the organic solvent, and more preferably contains only acetonitrile as the organic solvent.

The concentration of the organic solvent in an elution reagent is not particularly limited, and is preferably 20% or more, 30% or more, or 40% or more. The concentration of the organic solvent is preferably 65% or less, 60% or less, or 55% or less. The concentration “%” of the organic solvent as used herein is all volume/volume % (v/v %).

A pH of the releasing agent is not particularly limited as long as it is a pH recognized as basic by those skilled in the art. The pH of the releasing agent is preferably 10.85 or more, 10.9 or more, 10.95 or more, 11.0 or more, 11.05 or more, 11.1 or more, 11.15 or more, 11.2 or more, 11.25 or more, 11.3 or more, 11.35 or more, or 11.4 or more. Most preferably, the releasing agent has a pH of 11.4 or more. As a result, the Aβ peptide can be released from the complex particularly efficiently. The pH of the releasing agent is preferably 14.0 or less, 13.5 or less, 13.0 or less, 12.5 or less, 12.4 or less, 12.35 or less, 12.3 or less, 12.25 or less, 12.2 or less, 12.15 or less, 12.1 or less, 12.05 or less, or 12.0 or less.

The pH of the releasing agent can be adjusted by an amount (or concentration) of basic substance added. When ammonia or a salt thereof is used as the basic substance, the molar concentration of ammonium ions in the releasing agent is preferably 5.29 mM or more, 10.57 mM or more, 26.43 mM or more, 52.85 mM or more, 105.71 mM or more, or 132.14 mM or more. The molar concentration is preferably 1586 mM or less, 1533 mM or less, or 1480 mM or less. For example, when the basic substance is ammonia, the concentration of ammonia in the releasing agent is preferably 0.01% or more, 0.02% or more, 0.05% or more, 0.1% or more, 0.2% or more, or 0.25% or more. The concentration is preferably 3% or less, 2.9% or less, or 2.8% or less. The concentration “%” of ammonia as used herein is all weight/weight % (w/w %).

The releasing agent may contain additives such as stabilizers to an extent that it does not affect the release of Aβ peptide from the complex and the measurement of Aβ peptide by mass spectrometry. Examples of the additive include bovine serum albumin (BSA), human albumin, egg white albumin, monosaccharides such as glucose, disaccharides such as maltose, sugar alcohols such as mannitol and sorbitol, amino acids such as glycine, and the like. The additive may be one type or two or more types.

The above measurement method may include washing of removing an unreacted free component that has not formed a complex between the complex forming and the releasing. This washing includes B/F (Bound/Free) separation. The unreacted free component is a component that does not constitute a complex, and examples thereof include antibodies that have not bound to an Aβ peptide. The washing method is not particularly limited, and in a case where the complex is formed on a solid phase, when the solid phase is a particle, the complex and the unreacted free component can be separated by recovering the particle by centrifugation or magnetic separation, and removing supernatant. When the solid phase is a container such as a microplate or a microtube, the complex and the unreacted free component can be separated by removing a liquid containing the unreacted free component. After removing the unreacted free component, the solid phase capturing the complex may be washed with a suitable aqueous medium such as PBS.

Then, in the above measurement method, the released Aβ peptide is measured by mass spectrometry. The mass spectrometry is not particularly limited as long as the released Aβ peptide can be measured, and a known ionization capable of measuring the Aβ peptide can be used. Examples of the ionization include electrospray ionization (ESI), atmospheric chemical ionization (APCI), matrix-assisted laser desorption ionization (MALDI), and the like. Among them, the ESI method is particularly preferred.

The mass spectrometer used in the mass spectrometry is not particularly limited, and the mass spectrometer can be appropriately selected from known mass spectrometers. Examples thereof include a quadrupole (Q) mass spectrometer, ion trap (IT) mass spectrometer, a flight time (TOF) mass spectrometer, a Fourier transform ion cyclotron resonance (FTICR) mass spectrometer, an IT-TOF type mass spectrometer, a Q-TOF type mass spectrometer, a triple quadrupole (QqQ) type mass spectrometer, and the like. Among them, it is preferable to measure using a mass spectrometer capable of MS/MS measurement, and more preferably to measure using a triple quadrupole mass spectrometer.

In mass spectrometry, liquid chromatography-mass spectrometry (LC-MS), which is a combination of a mass spectrometer and a liquid chromatography apparatus, is preferably used, and LC-MS/MS which is a combination of liquid chromatography with a mass spectrometer capable of MS/MS measurement is preferably used.

The liquid chromatography apparatus is not particularly limited as long as it can be connected to a mass spectrometer, and a commercially available High Performance Liquid Chromatography (HPLC) apparatus can be used. The column connected to the liquid chromatography apparatus is not particularly limited, and a commercially available column for HPLC can be used. In this embodiment, the Aβ peptide released in the releasing agent can be subjected to a liquid chromatography apparatus. Therefore, pre-measurement process such as solvent exchange is not required.

The column is not particularly limited, but for example, a reversed-phase column can be used. Examples of a filler of the reversed-phase column include a silica-based filler, a polymer-based filler, and the like. Among them, a silica-based filler is preferred. As the reversed-phase column having a silica-based filler, a basic-resistant ODS column is more preferred.

As the solvent (mobile phase) used for liquid chromatography, a solution having the same composition as the releasing agent or a basic solution used for the releasing agent can be used. The concentration of organic solvent in the mobile phase is not particularly limited, and the concentration can be appropriately set according to measurement conditions. Liquid chromatography may be performed using an isocratic method using a mobile phase with a single concentration. Alternatively, liquid chromatography may be performed using a stepwise method or a gradient method in which a plurality of mobile phases with different compositions are used in combination.

Flow velocity of the mobile phase in the measurement can be appropriately set according to properties such as materials of the apparatus and the column and pressure resistance. The flow velocity of the mobile phase is preferably set to a flow velocity at which mass spectrometry is appropriately performed. The column temperature, the amount of a sample introduced into a measuring apparatus and the like can be appropriately set according to the measuring apparatus and the column.

In the above measurement method, measurement may be performed by a liquid chromatography apparatus to acquire information about the sample. Examples of the detector that performs measurement include a UV detector, a fluorescence detector, a differential refractive index detector, an electrical conductivity detector, an electrochemical detector, and the like.

The measurement of Aβ peptide by mass spectrometry can be appropriately set from a known technique according to the ionization and the mass spectrometer to be used. Among them, when a triple quadrupole mass spectrometer is used, it is preferable that the Aβ peptide is measured by multiple reaction monitoring (MRM) measurement in which the measurement mode is set to positive ion measurement mode.

The triple quadrupole mass spectrometer has a first quadrupole (Q1) and a third quadrupole (Q3) in front of and behind a collision cell (Q2). An object to be measured is ionized by an ion source to be precursor ions, and only ions having a specific mass-to-charge ratio (m/z) set in Q1 pass through a filter and are introduced into Q2. The precursor ions with a specific mass-to-charge ratio introduced into Q2 collide with an inert gas and cleave to be product ions. The product ions sent from Q2 to Q3 are filtered again in Q3, only ions with a specific mass pass through the filter and are sent from Q3 to a detector, and a signal is detected. As a result, a specific product ion for a specific precursor ion is detected by the detector. A combination of the specific mass-to-charge ratio set in Q1 and the specific mass-to-charge ratio set in Q3 is called an MRM transition. In MRM measurement, by setting multiple MRM transitions, multiple product ions can be detected at the same time. As a result, a plurality of substances contained in the object to be measured can be measured at the same time. For example, each MRM transition is set for a sample containing Aβ40 and Aβ42, whereby two types of Aβ peptides can be measured at the same time and each measured value can be acquired.

In the MRM measurement, the measurement mode is not particularly limited, but it is preferable to use the mass spectrometer in the positive ion measurement mode. Cone voltage and collision energy in the MRM measurement are also not particularly limited. The cone voltage and collision energy can be set to appropriate conditions by those skilled in the art.

For example, as an MRM transition for Aβ40, precursor ion/product ion can be set to 1083/1953.6. As an MRM transition for Aβ42, precursor ion/product ion can be set to 1129/1078.5. By setting the MRM transitions as described above, the Aβ peptide can be detected with high sensitivity.

In the above measurement method, the blood sample may be mixed with a labeled internal standard substance, and the measured value of the Aβ peptide may be acquired based on the measured value of the internal standard substance. The internal standard substance is not particularly limited as long as it can quantify the Aβ peptide, but the internal standard substance is preferably an Aβ peptide labeled with a stable isotope. Examples of the Aβ peptide labeled with a stable isotope include Aβ40 and Aβ42 labeled with N15 stable isotope. The amount of the internal standard substance added is not particularly limited as long as the concentration can be measured by the measuring apparatus. The amount of the internal standard substance added can be appropriately set according to analytical ability of the measuring apparatus.

In addition to the MRM transition for detecting the Aβ peptide, an MRM transition can be set and measured for the Aβ peptide labeled with a stable isotope as the internal standard substance, and the measured value can be acquired. As the MRM transition for Aβ40 labeled with N15 stable isotope, precursor ion/product ion can be set to 1096/1066.5. As the MRM transition for Aβ42 labeled with N15 stable isotope, precursor ion/product ion can be set to 1142.5/1091.5.

When an Aβ peptide in a blood sample is measured with a fully automated immunoassay system using the antibody set of this embodiment, the Aβ peptide can be measured with the same quantitative properties as when the blood sample is measured by the above measurement method. Specifically, correlation coefficient r calculated when measured value X of Aβ peptide obtained by measurement with a fully automated immunoassay system using the capture antibody and the detection antibody of the antibody set of this embodiment and measured value Y of Aβ peptide obtained by measurement by mass spectrometry are subjected to linear regression analysis is 0.8 or more, preferably 0.85 or more, and more preferably 0.9 or more. Procedure for calculating the correlation coefficient r will be described below.

Measurements with a fully automated immunoassay system include measuring an Aβ peptides in a blood sample using a capture antibody and a detection antibody labeled with alkaline phosphatase (ALP). Generally, in measurement with a fully automated immunoassay system, a reagent containing a capture antibody, a reagent containing a magnetic particle as a solid phase, a reagent containing an ALP-labeled detection antibody, and a reagent containing an ALP substrate solution are used. In a preferred embodiment, the capture antibody is previously labeled with a biotin and the magnetic particle is previously immobilized with an avidin. Alternatively, a reagent containing a capture antibody immobilized on the magnetic particle, a reagent containing an ALP-labeled detection antibody, and a reagent containing an ALP substrate solution are used. In a preferred embodiment, a reagent containing a buffer solution for diluting the blood sample and a reagent containing a buffer solution for promoting a reaction between ALP and a substrate (hereinafter, also referred to as a measurement buffer solution) may be further used. The buffer solution for diluting the blood sample is the same as described for the measurement method of this embodiment described above. The measurement buffer solution is a buffer solution containing metal ions necessary for an enzymatic reaction of ALP and having a pH and salt concentration suitable for the reaction. Examples of the measurement buffer solution include R4 reagent (Sysmex Corporation) and the like. When these reagents are provided in a fully automated immunoassay system, a blood sample containing an Aβ peptide is set and measurement is started, the Aβ peptide in the blood sample is measured by the sandwich ELISA described above. The number of the blood sample may be one or more.

In the measurement by mass spectrometry, the same blood sample as the measurement with a fully automated immunoassay system is measured. The measurement by mass spectrometry includes immunoprecipitating the Aβ peptide in the blood sample with 6E10 antibody which is an anti-Aβ monoclonal antibody, releasing the Aβ peptide from a complex of the immunoprecipitated Aβ peptide and the antibody by a basic solution containing an organic solvent, separating the solution containing the released Aβ peptide by liquid chromatography, and ionizing the separated Aβ peptide and measuring by a triple quadrupole mass spectrometer. Details of the measurement by mass spectrometry method are the same as those described for the measurement method of this embodiment.

In a preferred embodiment, the measured value X is acquired using HISCL (registered trademark)-5000 (Sysmex Corporation), and as mass spectrometry, the measured value Y is acquired by treatment using a predetermined releasing agent, LC separation using a predetermined column, and measurement using a predetermined mass spectrometer. That is, correlation coefficient r calculated when measured value X of Aβ peptide obtained by measurement by HISCL (registered trademark)-5000 using the capture antibody and the detection antibody of the antibody set of this embodiment and measured value Y of Aβ peptide obtained by measurement by mass spectrometry are subjected to linear regression analysis is 0.8 or more, preferably 0.85 or more, and more preferably 0.9 or more. The measurement by HISCL (registered trademark)-5000 include measuring an Aβ peptides in a blood sample using a capture antibody and a detection antibody labeled with alkaline phosphatase. Specifically, the measurement is as follows. In the measurement by HISCL (registered trademark)-5000, an R1 reagent containing a biotin-labeled capture antibody, an R2 reagent containing a streptavidin-immobilized magnetic particle, an R3 reagent containing an ALP-labeled detection antibody, an R4 reagent containing a measurement buffer solution and an R5 reagent containing an ALP substrate solution are used. When these reagents are provided in HISCL (registered trademark)-5000, a blood sample containing an Aβ peptide is set, and measurement is started, the blood sample and the R1 reagent are first mixed, and a complex of the Aβ peptide and the biotin-labeled capture antibody is formed. The R2 reagent is added thereto, and the complex is formed on the magnetic particle through a binding between biotin and streptavidin. The magnetic particle are magnetically collected to remove the liquid, and the magnetic particle is washed with a HISCL (registered trademark) washing liquid (B/F separation). The R3 reagent is added to the magnetic particle to form a complex of the Aβ peptide, the biotin-labeled capture antibody and the ALP-labeled detection antibody on the magnetic particle. B/F separation is performed in the same manner as above, the R4 reagent and the R5 reagent are added to the magnetic particle, chemiluminescence intensity is measured, and the measured value X is acquired.

In the measurement by mass spectrometry, the same blood sample as the measurement with a fully automated immunoassay system is measured. In a preferred embodiment, the measurement by mass spectrometry includes immunoprecipitating the Aβ peptide in the blood sample with 6E10 antibody which is an anti-Aβ monoclonal antibody, releasing the Aβ peptide from a complex of the immunoprecipitated Aβ peptide and the antibody by a solution containing 0.56% ammonia and 40% acetonitrile, separating the solution containing the released Aβ peptide by liquid chromatography with ACQUITY (registered trademark) UPLC (registered trademark) H-class biosystem using ACQUITY (registered trademark) UPLC (registered trademark) peptide BEH Cis column, and measuring the separated Aβ peptide using Xevo (registered trademark) TQ-XS triple quadrupole mass spectrometer using electrospray ionization. The measurement by mass spectrometer is a multiple reaction monitoring (MRM) measurement. In this MRM measurement, the mass spectrometer is used in the positive ion measurement mode, and MRM transitions are precursor ion/product ion of 1083.4/1054 for Aβ40 peptide and precursor ion/product ion of 1129.5/1078.8 for Aβ42 peptide. The blood sample is measured by the mass spectrometry to acquire measured value Y.

Both the measured value X with a fully automated immunoassay system and the measured value Y by mass spectrometry may be a signal intensity, or may be an amount or concentration value of the Aβ peptide acquired from a calibration curve or the like. In the calculation of the correlation coefficient r, the measured values of Aβ peptides acquired from the blood samples by each measurement method may be plotted on an XY plane in which the measured value X with a fully automated immunoassay system is taken on an X-axis and the measured value Y by mass spectrometry is taken on a Y-axis to acquire a regression line. The correlation coefficient r can be calculated by a known linear regression analysis. Examples of the linear regression analysis include a least squares method. In this embodiment, the correlation coefficient r is preferably 0.85 or more, and more preferably 0.9 or more. The calculation of the correlation coefficient itself can be performed by software such as Excel (registered trademark) (Microsoft Corporation).

A further embodiment is a method for measuring an Aβ peptide in vitro by an immunoassay using an antibody set including a capture antibody and a detection antibody that specifically bind to the Aβ peptide. This immunoassay is characterized by using an antibody that binds to an epitope including an N-terminal residue of the Aβ peptide as a capture antibody that specifically binds to the Aβ peptide, and using an antibody that binds to an epitope different from the epitope to which the capture antibody binds as a detection antibody that specifically binds to the Aβ peptide. In this embodiment, the Aβ peptide is at least one of Aβ40 or Aβ42. In this embodiment, it is preferable to use the above antibody set.

By using the above capture antibody and detection antibody, the immunoassay can measure an Aβ peptide with the same quantitative properties as the method for measuring an Aβ peptide of this embodiment using mass spectrometry. Specifically, the antibody set used in the immunoassay is an antibody set in which correlation coefficient r calculated when measured value X of Aβ peptide obtained by measurement with a fully automated immunoassay system using the capture antibody and the detection antibody and measured value Y of Aβ peptide obtained by measurement by mass spectrometry are subjected to linear regression analysis is 0.8 or more. In a more preferred embodiment, the antibody set used in the immunoassay is an antibody set having a correlation coefficient r of 0.85 or more, and a further preferred embodiment is an antibody set having a correlation coefficient r of 0.9 or more. Details of the measurement with a fully automated immunoassay system and mass spectrometry are the same as those described for the antibody set of this embodiment.

In a further embodiment, the antibody set used in the immunoassay is an antibody set in which correlation coefficient r calculated when measured value X of Aβ peptide obtained by measurement by HISCL (registered trademark)-5000 using the capture antibody and the detection antibody and measured value Y of Aβ peptide obtained by measurement by mass spectrometry by treatment using a predetermined releasing agent, LC separation using a predetermined column and measurement using a predetermined mass spectrometer are subjected to linear regression analysis is 0.8 or more. In a more preferred embodiment, the antibody set used in the immunoassay is an antibody set having a correlation coefficient r of 0.85 or more, and a further preferred embodiment is an antibody set having a correlation coefficient r of 0.9 or more. Details of the measurement by HISCL (registered trademark)-5000 and mass spectrometry are the same as those described for the antibody set of this embodiment.

A further embodiment is a reagent kit for measuring an Aβ peptides. That is, a reagent kit for measuring an Aβ peptide (hereinafter, also referred to as “reagent kit”) including the capture antibody and the detection antibody of the antibody set of this embodiment described above is provided. The capture antibody and the detection antibody are as described above. The reagent kit of this embodiment includes one or more reagents.

The reagent kit of this embodiment may include a basic solution containing an organic solvent for releasing an Aβ peptide from a complex in which the Aβ peptide and the capture antibody are bound. The basic solution containing an organic solvent is as described above.

The detection antibody may be labeled with a labeling substance. The labeling substance is not particularly limited, but the labeling substance is preferably an enzyme. Alternatively, the reagent kit may further include a labeling substance for labeling the detection antibody. When the labeling substance is an enzyme, the reagent kit may further contain a substrate for the enzyme. The labeling substance and the substrate are as described above. Forms of the capture antibody, the detection antibody, the labeling substance and the substrate are not particularly limited, and they may be a solid (for example, powder, crystal, freeze-dried product, and the like) or liquid (for example, solution, suspension, emulsion, and the like).

In this embodiment, a container containing each reagent may be packed in a box and provided to a user. The box may contain an attached document. The attached document may describe a composition of the reagent kit, method of use, relationship between the measurement result of a fully automated immunoassay system obtained by the reagent kit and the measurement result by mass spectrometer, and the like. Examples of the reagent kit are shown in some figures below. However, this embodiment is not limited to these examples.

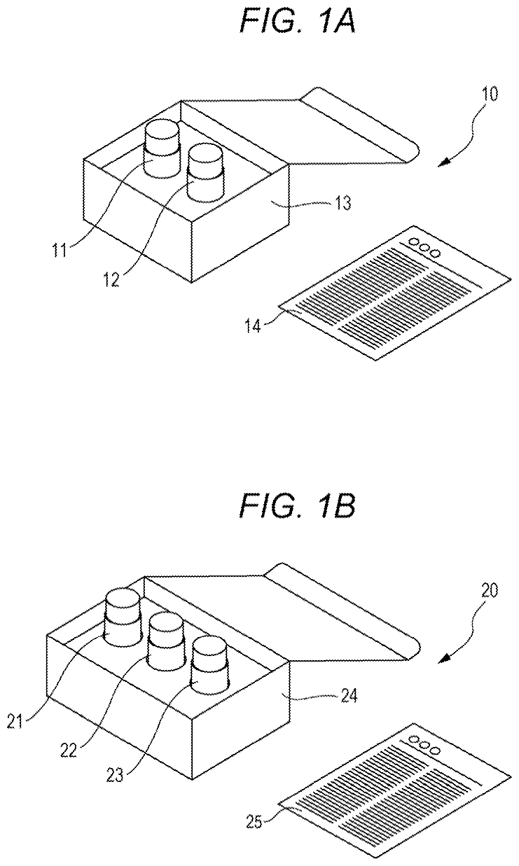

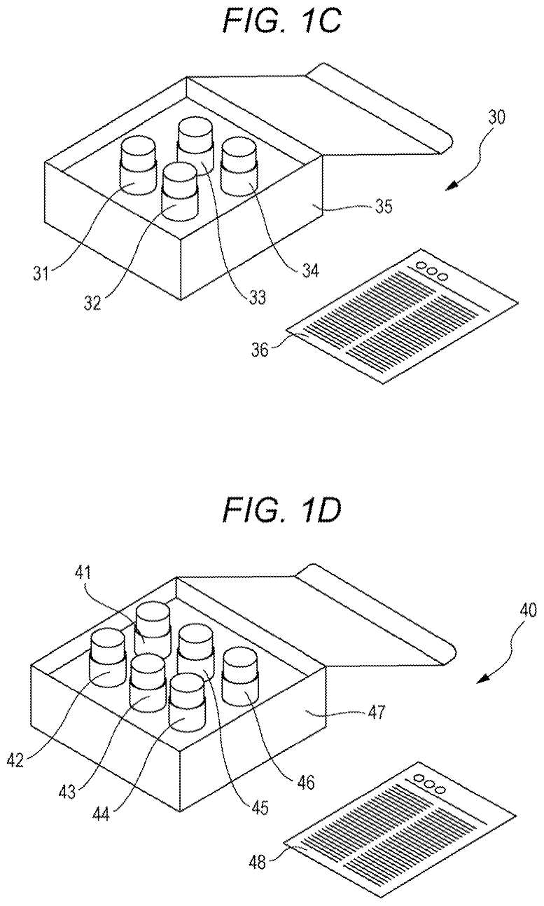

FIG. 1A shows an example of the reagent kit of this embodiment. In FIG. 1A, 10 denotes a reagent kit, 11 denotes a first container containing a reagent containing a capture antibody for Aβ peptide, 12 denotes a second container containing a reagent containing a detection antibody for Aβ peptide, 13 denotes a packing box, and 14 denotes an attached document. In this example, the reagent kit may further include a solid phase for immobilizing the capture antibody. The solid phase is as described above.

FIG. 1B shows an example of a reagent kit of a further embodiment. In FIG. 1B, 20 denotes a reagent kit, 21 denotes a first container containing a reagent containing a capture antibody for Aβ peptide, 22 denotes a second container containing a reagent containing a detection antibody for Aβ40, 23 denotes a third container containing a reagent containing a detection antibody for Aβ42, 24 denotes a packing box, and 25 denotes an attached document. In this example, the reagent kit may further include a solid phase for immobilizing the capture antibody. Details of the solid phase are as described above.

FIG. 1C shows an example of a reagent kit of a further embodiment. In FIG. 1C, 30 denotes a reagent kit, 31 denotes a first container containing a reagent containing a capture antibody for Aβ40 peptide, 32 denotes a second container containing a reagent containing a detection antibody for Aβ40, 33 denotes a third container containing a reagent containing a detection antibody for Aβ42, 34 denotes a fourth container containing a reagent containing a basic solution containing an organic solvent, 35 denotes a packing box, and 36 denotes an attached document. In this example, the reagent kit may further include a solid phase for immobilizing the capture antibody. The solid phase is as described above.

FIG. 1D shows an example of a reagent kit of a further embodiment. In FIG. 1D, 40 denotes a reagent kit, 41 denotes a first container containing a reagent containing a capture antibody for Aβ peptide, 42 denotes a second container containing a reagent containing an enzyme-labeled detection antibody for Aβ40, 43 denotes a third container containing a reagent containing an enzyme-labeled detection antibody for Aβ42, 44 denotes a fourth container containing a reagent containing a magnetic particle, 45 denotes a fifth container containing a reagent containing a washing reagent, 46 denotes a sixth container containing an enzyme substrate, 47 denotes a packing box, and 48 denotes an attached document.

The reagent kit of this embodiment may include a calibrator for Aβ peptide. Examples of the calibrator include a calibrator for quantifying Aβ40 and a calibrator for quantifying Aβ42. The Aβ40 calibrator may include, for example, a buffer solution containing no Aβ40 (negative control) and a buffer solution containing Aβ40 at a known concentration. Another example of the calibrator includes a buffer solution containing neither Aβ40 nor Aβ42 (negative control), a buffer solution containing Aβ40 at a known concentration, and a buffer solution containing Aβ42 at a known concentration. Another example of the calibrator includes a buffer solution containing neither Aβ40 nor Aβ42 (negative control), a buffer solution containing Aβ40 and Aβ42 at known concentrations, respectively.

Hereinafter, the present disclosure will be described more specifically with reference to Examples.

### EXAMPLES

### Example 1: Measurement of Plasma Aβ Peptide Using Combination of Immunoprecipitation Using Basic Solution Containing Organic Solvent and Mass Spectrometry

An Aβ peptide was released from a complex of an Aβ peptide and an antibody that specifically binds to the Aβ peptide using a basic solution containing an organic solvent, and the Aβ peptide was measured using mass spectrometry.

(1) Capture and Release of Aβ Peptide Using Immunoprecipitation

(1.1) Blood Sample

As blood samples containing an Aβ peptide, 5 types of commercially available plasma samples (ProMedeX) from different lots were used.

(1.2) Antibody that Specifically Binds to Aβ Peptide

As an antibody that specifically binds to the Aβ peptide, 6E10 antibody (BioLegend, Inc.) which is a commercially available mouse monoclonal anti-Aβ antibody was used. The 6E10 antibody was immobilized on a magnetic particle (M-270 Epoxy-activated Dynabeads: Thermo Fisher Scientific Inc.) by a conventional method.

(1.3) Aβ Peptide

An Aβ40 peptide and an Aβ42 peptide were purchased from AnaSpec, Inc. for preparation of a calibration curve. As internal standard substances, 15N and 15N-Aβ40 and 15N-Aβ42 (rPeptide) which were an Aβ40 peptide and an Aβ42 peptide each labeled with a stable isotope ¹⁵N were used. The Aβ40 peptide was suspended in PBS solutions containing 3% BSA, so as to have final concentrations of 10.8 pg/ml, 21.7 pg/ml, 43.3 pg/ml, 86.6 pg/ml, 173.2 pg/ml, 346.4 pg/ml and 692.8 pg/ml, respectively. The Aβ42 peptide was suspended in PBS solutions containing 3% BSA, so as to have final concentrations of 2.8 pg/ml, 5.6 pg/ml, 11.3 pg/ml, 22.6 pg/ml, 45.2 pg/ml, 90.3 pg/ml and 180.6 pg/mL, respectively. 15N-Aβ40 and 15N-Aβ42 were suspended in the same solution in PBS solutions containing 3% BSA so as to be 500 pg/ml, respectively.

(1.4) Preparation of Basic Solution Containing Organic Solvent

As a basic solution (releasing agent) containing an organic solvent, 1.2 ml of 28% concentrated ammonia water (Nacalai Tesque, Inc.) and 6.0 ml of acetonitrile (Kanto Chemical Co., Inc.) were added and mixed to 12.8 ml of pure water to prepare a 1.68% ammonia/30% acetonitrile solution.

(1.5) Immunoprecipitation

A 250 μl of plasma sample or each of the Aβ40 peptide solutions or Aβ42 peptide solution prepared in (1.3) above was added to a 1.5 ml sample tube (Eppendorf AG). To each sample tube containing the above solution was added 250 μl of the solution containing 15N-Aβ40 and 15N-Aβ42 prepared in (1.3) above, and the sample tube was allowed to stand at room temperature for 30 minutes. After standing the sample tube, 40 μl of the suspension of magnetic particles (4 pg antibody/0.4 mg magnetic particles) immobilized with 6E10 antibody prepared in (1.2) above was added to each sample solution, and the mixture was inverted and mixed for 1 hour using a rotator at room temperature to form a complex of the Aβ peptide and the antibody. These solutions were focused using a magnetic stand to remove supernatant.

(1.6) Washing

After removing the supernatant, 1 mL of a PBS solution containing 3% BSA was added to the magnetic particles remaining in the sample tube, mixed, and then magnetized again to remove supernatant. This operation was performed twice with 1 mL of the PBS solution containing 3% BSA, twice with 1 mL of a 50 mM ammonium acetate solution and once with 1 mL of ultrapure water successively to wash the magnetic particles.

(1.7) Release of Aβ Peptide from Complex

After washing the magnetic particles in (1.6) above, 25 μL of the releasing agent prepared in (1.4) above was added to the remaining magnetic particles after removing the washing liquid, mixed, and the mixture was allowed to stand for 1 minute. The magnetic particles were magnetically collected again, and supernatant was recovered as an eluate.

(2) Mass Spectrometry

The eluate prepared in (1.7) above was subjected to LC-MS/MS for MRM measurement, and the Aβ peptide was measured. ACQUITY (registered trademark) UPLC (registered trademark) H-class biosystem (Waters Corporation: hereinafter also referred to as UPLC) was used for a liquid chromatography section of LC-MS/MS. As the column, an ACQUITY (registered trademark) UPLC (registered trademark) peptide BEH C₁₈ column (Waters Corporation) which is a reversed-phase column was used. As the mass spectrometer, Xevo (registered trademark) TQ-XS triple quadrupole mass spectrometer (Waters Corporation: hereinafter also referred to as TQ-XS mass spectrometer) was used.

Each eluate was placed on a UPLC autosampler, and 10 μl of the eluate was introduced into the UPLC and fractionated by gradient. Conditions for the gradient were as follows.

TABLE 1

| Analysis apparatus | Xevo TQ-XS triple quadrupole mass spectrometer | |
|---|---|---|
| Column | ACQUITY UPLC Peptide BEH C₁₈ column(300 | |
| | Å, 1.7 μm, 2.1 mm × 150 mm) | |
| Introduction amount | 10 μl | |
| Flow velocity | 200 μl/min | |
| Temperature | 50° C. | |
| Mobile phase A | 0.1% ammonia solution | |
| Mobile phase B | 0.01% ammonia, 90% acetonitrile solution | |
| Gradient conditions | 0 to 0.1 min | 90% A, 10% B |
| | 1.0 to 5.5 min | 90-45% A, 10-55% B |
| | 5.5 to 6.7 min | 45% A, 55% B |
| | 6.7 to 7.0 min | 45-90% A, 55-10% B |
| | 7.0 to 8.5 min | 90% A, 10% B |

An eluate that was subjected to the gradient and eluted from the column was directly subjected to the TQ-XS mass spectrometer connected to the UPLC. The TQ-XS mass spectrometer used electrospray ionization and measured in positive ion mode. Conditions for MRM measurement were set as shown in Table 2 below.

TABLE 2

| | Precursor ion | Product ion | Cone voltage | Collision energy |
|---|---|---|---|---|
| | (m/z) · 4+ | (m/z) · 4+ | (V) | (eV) |
| Aβ40 | 1083.4 | 1054.0 | 32 | 22 |
| Aβ42 | 1129.5 | 1078.8 | 28 | 25 |
| 15N-Aβ40 | 1096 | 1066.5 | 32 | 22 |
| 15N-Aβ42 | 1142.6 | 1091.5 | 28 | 25 |

(3) Measurement Results

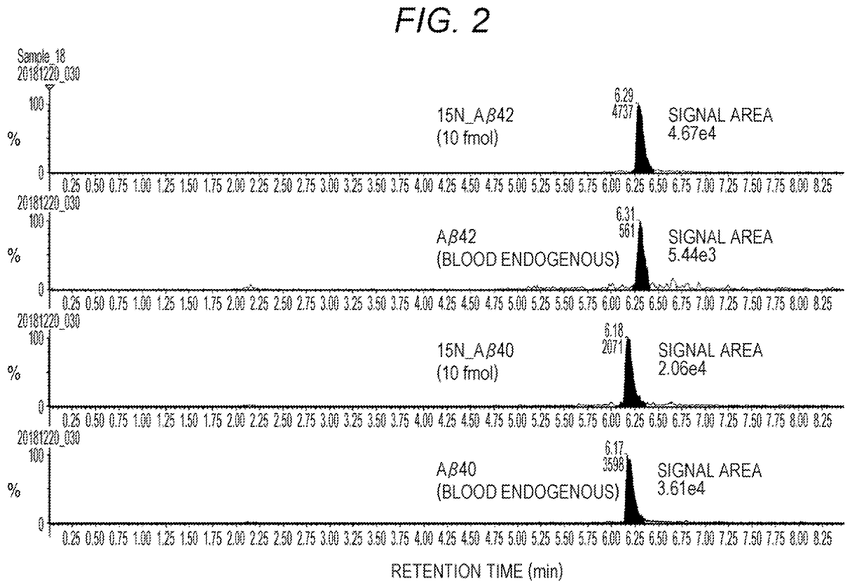

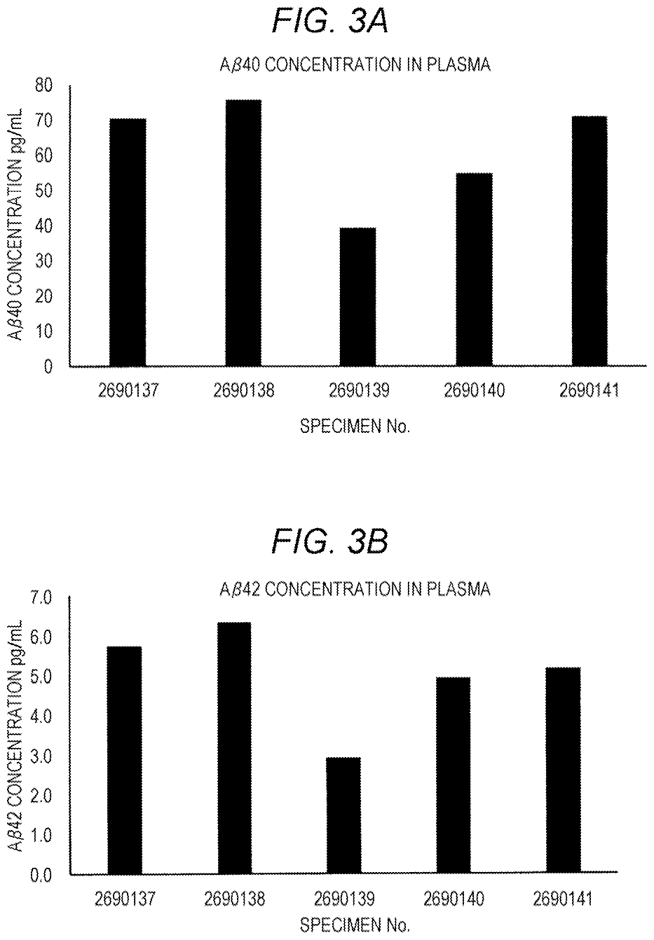

Measurement results using LC-MS/MS are shown in FIGS. 2, 3A and 3B. FIG. 2 shows results of MRM measurement for Aβ40 peptide, Aβ42 peptide, 15N-Aβ40 and 15N-Aβ42, and FIGS. 3A and 3B show concentrations of Aβ40 peptide and Aβ42 peptide in the measured plasma specimens. From these results, it was shown that the Aβ peptide can be released from the complex using a basic solution containing an organic solvent and the Aβ peptide can be measured by mass spectrometry.

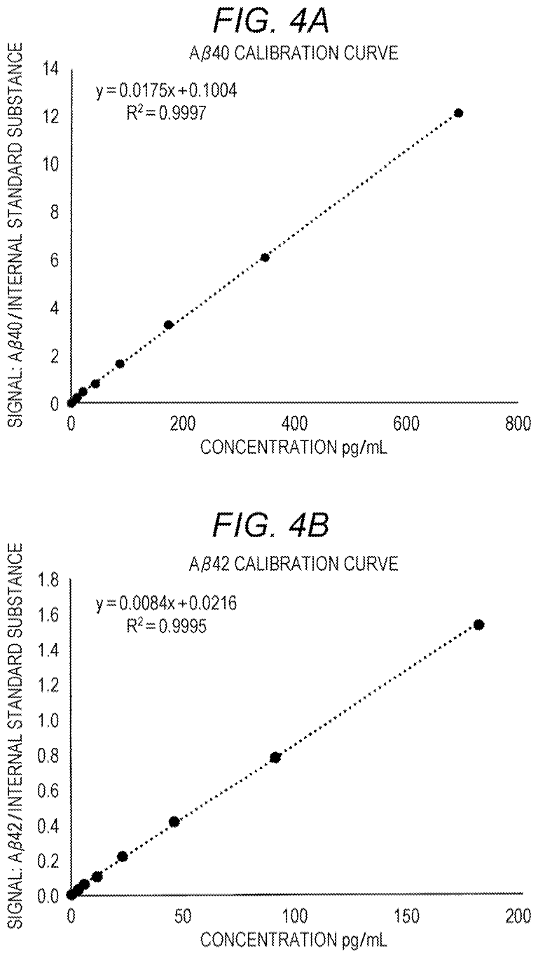

FIGS. 4A and 4B show results of preparing a calibration curve based on the measured values detected using the Aβ peptide with a known concentration prepared in (1.3) above. As shown in FIGS. 4A and 4B, it was possible to prepare a calibration curve in which R² was 0.999 or more in both the Aβ40 peptide and the Aβ42 peptide. Low concentrations of Aβ peptide of 100 pg/ml or less were also detectable. From this, it was shown that the above measurement method which is a combination of a basic solution containing an organic solvent and mass spectrometry can measure Aβ peptide with high sensitivity and has excellent quantitative properties.

### Example 2: Comparison of Elution Efficiency

The composition of the releasing agent was changed, and an elution efficiency of Aβ peptide from the complex due to difference in the composition of the releasing agent was calculated based on the following calculation formula and compared.

[Elution efficiency (%)]=[Aβ peptide concentration of sample C]/([Aβ peptide concentration of sample A]−[Aβ peptide concentration of sample B])×100

(1) Comparison of Basic Substances in Releasing Agents

(1.1) Preparation of Releasing Agent

DDM (n-dodecyl-β-D-maltoside, Sigma-Aldrich Co. LLC.), acetonitrile and/or 28% concentrated ammonia water were appropriately selected and mixed with pure water so as to have the compositions shown in Table 3 below to prepare various releasing agents. A solution containing no basic substance or organic solvent was also referred to as a releasing agent for convenience. After preparation, pH of the solution containing the basic substance was measured using a pH meter (HORIBA, Ltd.).

TABLE 3

| | Releasing agent composition | pH |
|---|---|---|
| Comparative reagent 1 | DDM, 70% acetonitrile | |
| Comparative reagent 2 | DDM, 0.056% ammonia | 11.013 |
| Comparative reagent 3 | DDM, 0.56% ammonia | 11.582 |
| Reagent 1 | 50% acetonitrile, 0.028% ammonia | 10.854 |
| Reagent 2 | 50% acetonitrile, 0.07% ammonia | 11.059 |
| Reagent 3 | 50% acetonitrile, 0.14% ammonia | 11.219 |
| Reagent 4 | 50% acetonitrile, 0.28% ammonia | 11.448 |
| Reagent 5 | 50% acetonitrile, 0.56% ammonia | 11.613 |
| Reagent 6 | 50% acetonitrile, 1.12% ammonia | 11.801 |
| Reagent 7 | 50% acetonitrile, 1.68% ammonia | 11.973 |
| Reagent 8 | 50% acetonitrile, 2.24% ammonia | 11.974 |
| Reagent 9 | 50% acetonitrile, 2.80% ammonia | 12.038 |

(1.2) Sample Preparation

Sample A was prepared by suspending the Aβ40 peptide used in (1.3) of Example 1 in a PBS solution containing 3% BSA so as to be 1000 pg/ml. The Aβ peptide concentration of sample A corresponds to an initial concentration of Aβ40 peptide.

(1.3) Immunoprecipitation

The sample A prepared in (1.2) above was immunoprecipitated in the same manner as in (1.5) of Example 1 to recover magnetic particles. At this time, supernatant after magnetization was collected and stored as sample B. An Aβ peptide concentration of sample B corresponds to a concentration of Aβ peptide that could not be captured by an antibody that specifically binds to the Aβ peptide.

(1.4) Washing/Elution

The magnetic particles recovered in (1.3) above were washed in the same manner as in (1.6) of Example 1, and a washing liquid was removed. To the washed magnetic particles was added 15 μl of each of the releasing agents prepared in (1.1) above, and the mixture was allowed to stand for 1 minute to release the Aβ peptide. After standing the mixture, the magnetic particles were magnetically collected and supernatant was collected. A pH neutralizing solution (pH 7.4) containing 300 mM Tris and 300 mM NaCl was mixed with the collected supernatant to obtain sample C. The Aβ peptide concentration of the sample C corresponds to the concentration of Aβ peptide released after capture by immunoprecipitation.

(2) Measurement by Immunoassay

For the above samples A, B and C, the Aβ peptide concentration in each sample was measured by an immunoassay using a fully automated immunoassay system HISCL (registered trademark)-5000 (Sysmex Corporation). An R1 reagent (capture antibody reagent) was prepared by labeling 82E1 antibody with biotin by a conventional method and dissolving it in a buffer at pH 7.5 containing 1% BSA, 0.1 M Tris-HCl, 0.15 M NaCl and 0.1% NaN₃. As an R2 reagent (solid phase), a HISCL (registered trademark) R2 reagent (Sysmex Corporation) containing streptavidin-bound magnetic particles was used. An R3 reagent (detection antibody reagent) was prepared by labeling 1A10 antibody with alkaline phosphatase (ALP) by a conventional method and dissolving it in a buffer at pH 7.5 containing 1% BSA, 0.1 M Tris-HCl, 0.15 M NaCl and 0.1% NaN₃. As an R4 reagent (measurement buffer solution), a HISCL R4 reagent (Sysmex Corporation) was used. As an R5 reagent (ALP substrate solution), a HISCL R5 reagent (Sysmex Corporation) was used.

Measurement procedure according to HISCL (registered trademark)-5000 was as follows. The sample A, B or C (30 μL) and the R1 reagent (110 μL) were mixed and reacted at 42° C. for 4 minutes. After the reaction, the R2 reagent (30 μL) was added, and the mixture was reacted at 42° C. for 3 minutes. The magnetic particles in the obtained mixed solution were magnetically collected, supernatant was removed, and a HISCL washing liquid (300 μL) was added to wash the magnetic particles. Supernatant was removed, and the R3 reagent (100 μL) was added to the magnetic particles and mixed, and the mixture was reacted at 42° C. for 5 minutes. The magnetic particles in the obtained mixed solution were magnetically collected, supernatant was removed, and a HISCL washing liquid (300 μL) was again added to wash the magnetic particles. Supernatant was removed, and the R4 reagent (50 μL) and the R5 reagent (100 μL) were added to the magnetic particles, and the chemiluminescence intensity was measured. As calibrators (antigens for preparing calibration curve), using each of solutions prepared by suspending the Aβ40 peptide in a solution at pH 7.0 containing 0.1% BSA, 0.14 M triethanolamine, 0.15 M NaCl and 0.1% NaN₃ so as to be 0 pg/ml, 8.6 pg/ml, 33.3 pg/ml, 99.2 pg/ml, 319.1 pg/ml and 1188.1 pg/ml, respectively, the same measurement was performed to prepare a calibration curve. The chemiluminescent intensity obtained by the measurement was applied to the calibration curve to determine the concentration of Aβ40 peptide.

(3) Measurement Results

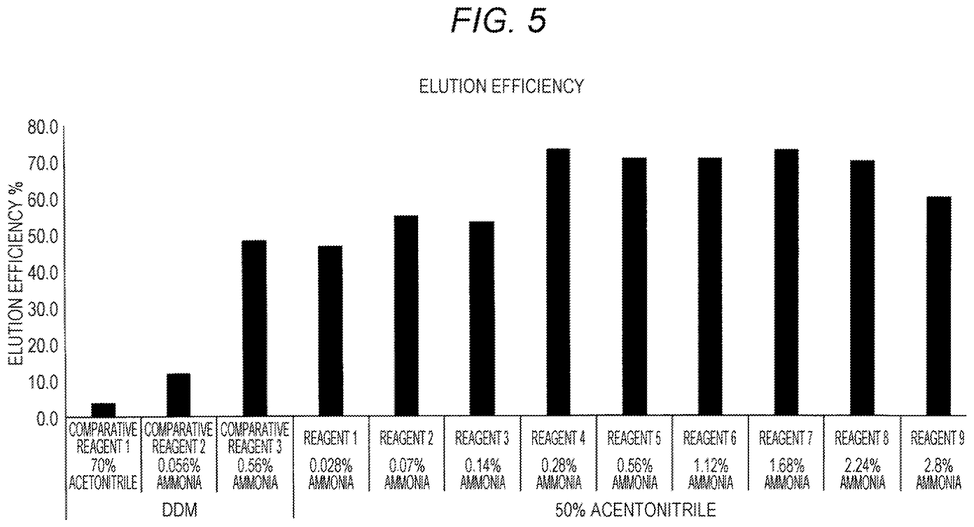

Measurement results of the elution efficiencies of comparative reagents 1 to 3 and reagents 1 to 9 in Table 3 are shown in FIG. 5. As shown in FIG. 5, it was shown that the elution efficiency is low with the releasing agent containing only the organic solvent or the basic solution, and high elution efficiency is obtained when the releasing agent containing the organic solvent and the basic substance is used as the releasing agent for the Aβ peptide. In particular, it was shown that reagents having a pH of 11.4 or more and 12.0 or less can obtain a high elution efficiency of more than 70%.

(4) Comparison of Releasing Agents for Organic Solvents

In (1.1) above, the elution efficiency was measured using the releasing agent shown in Table 4 below instead of the releasing agent shown in Table 3. In comparative reagent 5 and reagents 17 to 23, an Aβ42 peptide was used as a sample instead of the Aβ40 peptide. When the Aβ42 peptide was used, H31L21 antibody was used instead of the 1A10 antibody as a detection antibody, and each of solutions prepared by suspending the Aβ42 peptide in a solution at pH 7.0 containing 0.1% BSA, 0.14 M triethanolamine, 0.15 M NaCl and 0.1% NaN₃ so as to be 0 pg/ml, 0.5 pg/ml, 6.1 pg/ml, 65.2 pg/ml and 804.5 pg/ml, respectively, was used as calibrators. Except for the above, the same operations as in (1) and (2) were carried out, and the elution efficiency of each releasing agent was measured.

TABLE 4

| | Releasing agent | | Releasing agent |
|---|---|---|---|
| | composition | | composition |
| Comparative | 0.56% Ammonia | Comparative | 0.56% Ammonia |
| reagent 4 | | reagent 5 | |
| Reagent 10 | 40% Acetonitrile | Reagent 17 | 40% Acetonitrile |
| | 0.56% Ammonia | | 0.56% Ammonia |
| Reagent 11 | 40% Acetone | Reagent 18 | 40% Acetone |
| | 0.56% Ammonia | | 0.56% Ammonia |
| Reagent 12 | 40% 2-Propanol | Reagent 19 | 40% 2-Propanol |
| | 0.56% Ammonia | | 0.56% Ammonia |
| Reagent 13 | 40% Hexane | Reagent 20 | 40% Hexane |
| | 0.56% Ammonia | | 0.56% Ammonia |
| Reagent 14 | 40% 1-Propanol | Reagent 21 | 40% 1-Propanol |
| | 0.56% Ammonia | | 0.56% Ammonia |
| Reagent 15 | 40% Ethanol | Reagent 22 | 40% Ethanol |
| | 0.56% Ammonia | | 0.56% Ammonia |
| Reagent 16 | 40% DMSO | Reagent 23 | 40% DMSO |
| | 0.56% Ammonia | | 0.56% Ammonia |

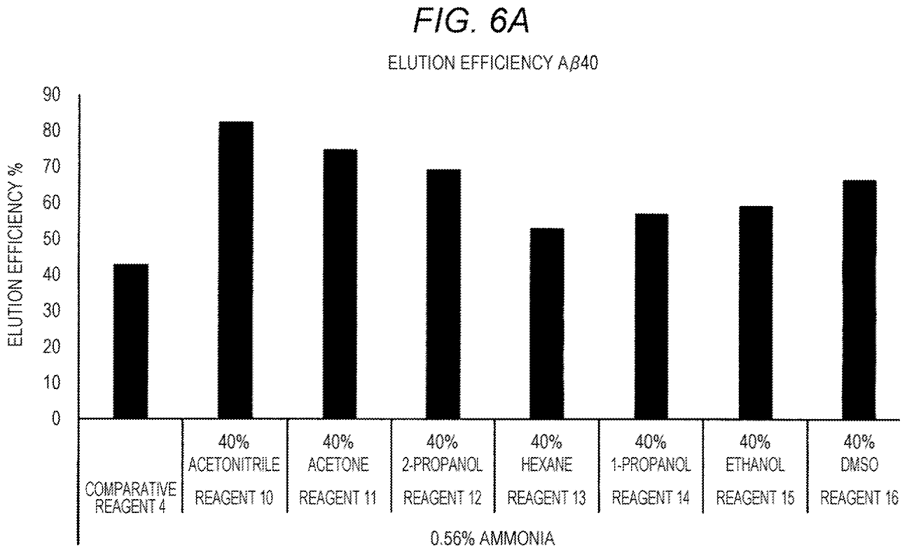

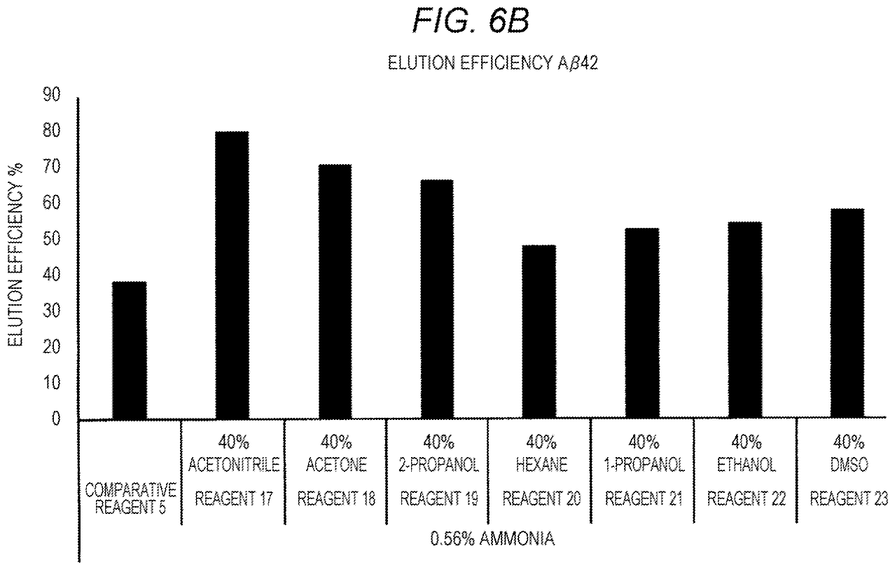

Measurement results for comparative reagent 4 and reagents 10 to 16 are shown in FIG. 6A, and measurement results for comparative reagent 5 and reagents 17 to 23 are shown in FIG. 6B. From FIGS. 6A and 6B, it was found that the reagents 10 to 16 and 17 to 23 showed better elution efficiency than comparative reagents 4 and 5 containing no organic solvent. It was shown that excellent elution efficiency was obtained when the releasing agent contained a basic substance and an organic solvent.

### Example 3: Carryover Measurement

Conventionally, an acidic solution is used when releasing an Aβ peptide from a complex of the Aβ peptide captured by an antibody and the antibody. Carryover of Aβ peptide to a liquid chromatography apparatus was measured under acidic and basic conditions of the releasing agent that elutes the complex.

(1) Sample Preparation

As an acidic releasing agent, formic acid (FUJIFILM Wako Pure Chemical Corporation) and acetonitrile were mixed with pure water to prepare an acidic releasing agent having a composition of 0.1% formic acid and 50% acetonitrile (here, an acidic solution is also referred to as a releasing agent for convenience). A releasing agent (1.68% ammonia, 50% acetonitrile) was prepared in the same manner as in (1.4) of Example 1. To these releasing agents was added the same Aβ40 peptide as in (1.3) of Example 1 so as to have a final concentration of 100 fmol/ul (Aβ40 peptide-containing solution). A solution containing no Aβ40 peptide (Aβ40 peptide-free solution) was also prepared for each of the acidic releasing agent and the basic releasing agent.

(2) Introduction of Sample into Liquid Chromatography

The four solutions prepared in (1) above were subjected to LC-MS/MS. As an LC-MS/MS apparatus and a column, those described in the mass spectrometry of (2) of Example 1 were used. Each solution was placed on the UPLC autosampler, and 10 μl of each solution was introduced into the UPLC and fractionated. UPLC analysis conditions were as shown in Table 5 below. Conditions for MRM measurement in mass spectrometry are shown in Table 2 of Example 1.

TABLE 5

| | |
|---|---|
| Analysis apparatus | Xevo TQ-XS triple quadrupole mass spectrometer |
| Column | ACQUITY UPLC Peptide BEH C₁₈ column(300 |
| | Å, 1.7 μm, 2.1 mm × 150 mm) |
| Introduction amount | 10 μl |
| Flow velocity | 200 μl/min |
| Temperature | 50° C. |
| Mobile phase A | 0.1% Ammonia solution |
| Mobile phase B | 0.01% Ammonia, 90% acetonitrile solution |
| Elution conditions | 50% A, 50% B |

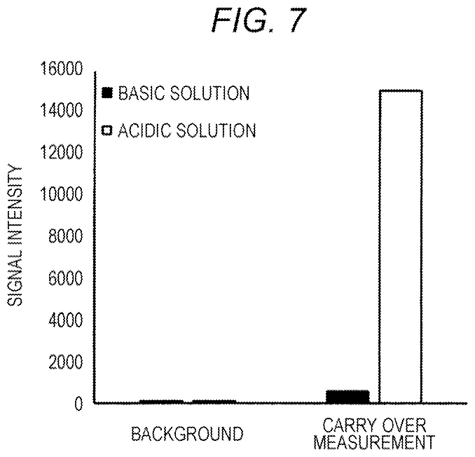

The two types of Aβ40 peptide-containing solutions prepared in (1) above and the corresponding Aβ40-free solutions thereof were subjected to UPLC, in the order of the Aβ40 peptide-free solution (background), Aβ40 peptide-containing solution, and Aβ40 peptide-free solution (carryover measurement). The results of comparing signal intensities obtained at that time are shown in FIG. 7. From FIG. 7, it was found that a large amount of carryover occurred in a case where the acidic releasing agent was used as compared with a case where the basic releasing agent was used. On the other hand, when the basic releasing agent was used as the releasing agent for Aβ peptide, carryover could be remarkably suppressed. Therefore, it was shown that it is appropriate to use a basic releasing agent for continuous analysis of Aβ peptide using mass spectrometry.

### Reference Example: Comparison of Releasing Agents for Acidity and Basicity

Carryover occurred when the Aβ peptide released using an acidic releasing agent was measured by LC-MS/MS as in Example 3 above. In this reference example, difference in the amount of Aβ peptide detected was compared between a case where a basic releasing agent is used when releasing the Aβ peptide from the complex, and a case where an acidic releasing agent is used when releasing the Aβ peptide, then replaced with a basic releasing agent and subjected to LC-MS/MS.

(1) Sample Preparation

As the Aβ peptide sample, the same Aβ40 peptide and Aβ42 peptide as in (1.3) of Example 1 were used, and a solution prepared so that the Aβ40 peptide was 50 pg/ml or 190 pg/ml in a 3% BSA solution, or a solution prepared so that the Aβ42 peptide was 26 pg/ml or 103 pg/ml in a 3% BSA solution was prepared and used. As the acidic releasing agent, trifluoroacetic acid (TFA, FUJIFILM Wako Pure Chemical Corporation) and acetonitrile were mixed with pure water to prepare an acidic solution having a composition of 0.1% TFA and 30% acetonitrile. As the basic releasing agent, a 1.68% ammonia and 50% acetonitrile solution was prepared in the same manner as in (1.4) of Example 1.

(2) Measurement

(2.1) Immunoprecipitation

An internal standard substance was added to each of the samples prepared in (1) above in the same manner as in (1.5) of Example 1, and magnetic particles immobilized with 6E10 antibody were added to form a complex.

(2.2) Elution of Aβ Peptide

Each complex prepared in (2.1) above was subjected to the same washing as in (1.6) of Example 1, and then 25 μL of the acidic releasing agent or basic releasing agent prepared in (1) above was added and mixed, and the mixture was allowed to stand for 1 minute. The magnetic particles were magnetically collected again, and supernatant was recovered as an eluate.

(2.3) Solvent Exchange

Of the eluates recovered in (2.2) above, the eluate obtained using the acidic releasing agent was dried under reduced pressure for 1 hour under the conditions of a rotation speed of 1500 r/min and a temperature of 55° C. using Spin Dryer Standard VC-96R (TAITEC CORPORATION) in SpinDryer mode to volatilize the solvent. To a residue after drying under reduced pressure, 25 μL of the basic releasing agent prepared in (1) above was added to resuspend the residue.

(2.4) Mass Spectrometry

The eluate (referred to as solution X) obtained by using each basic releasing agent recovered in (2.2) above and the solution (referred to as solution Y) resuspended in (2.3) above were subjected to LC-MS/MS in the same manner as in (2) of Example 1, and MRM measurement was performed.

(3) Measurement Results

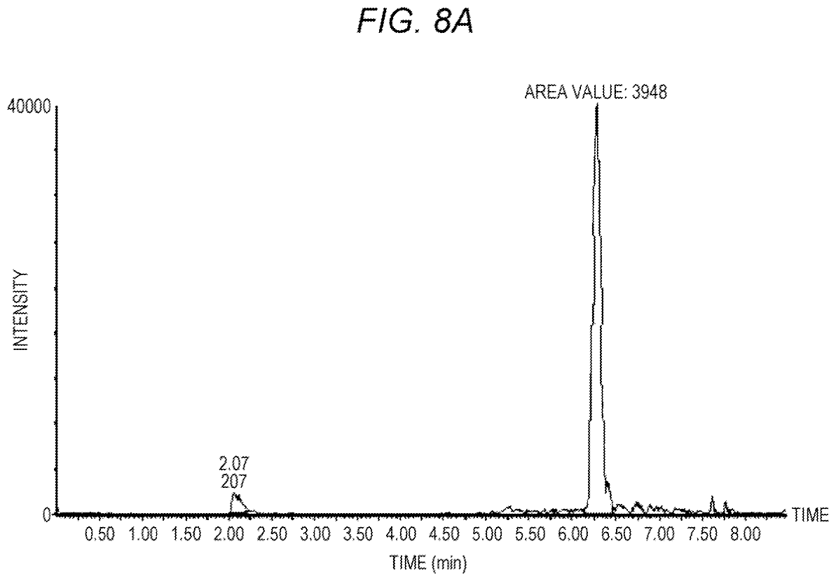

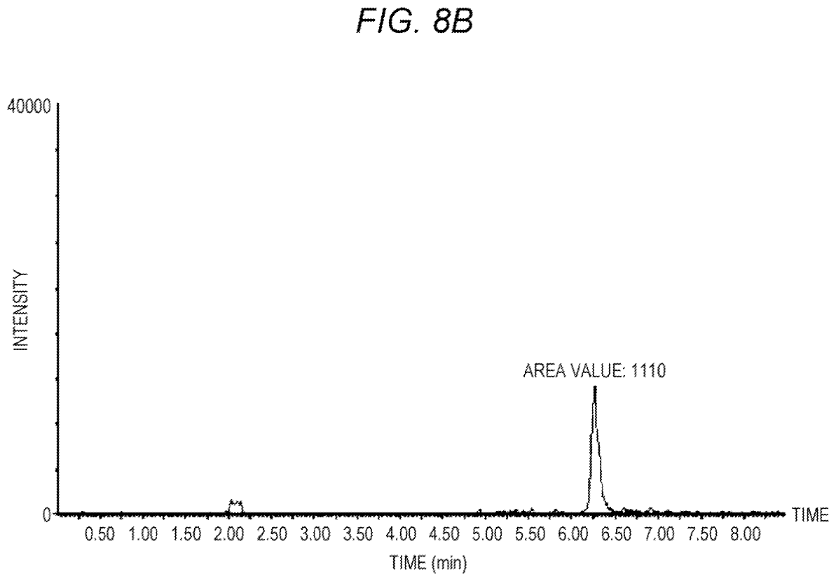

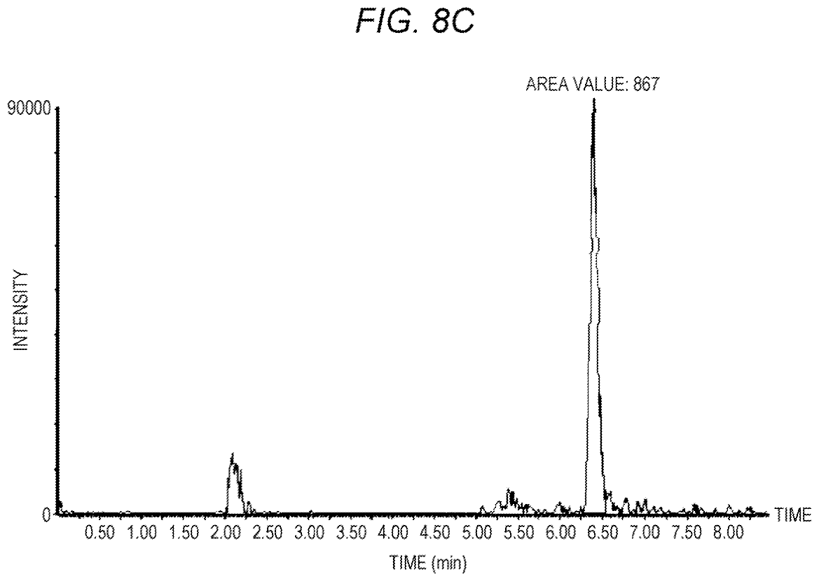

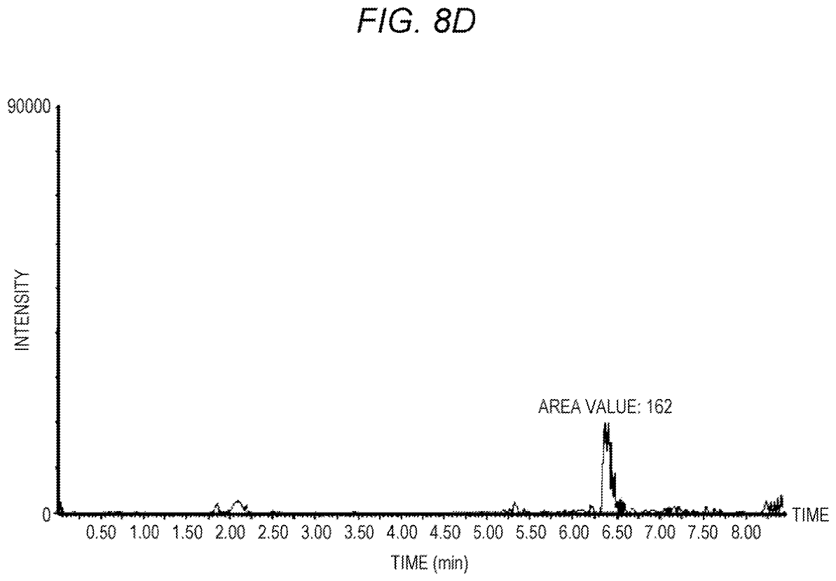

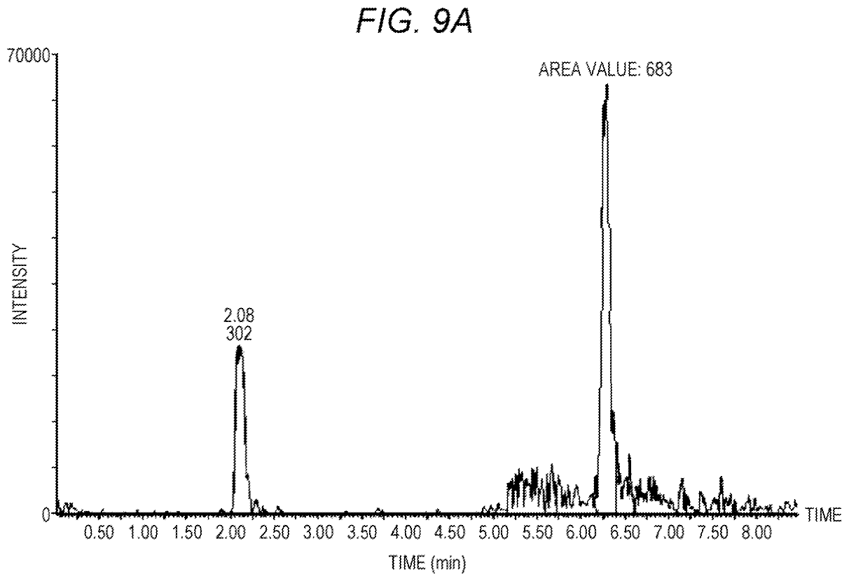

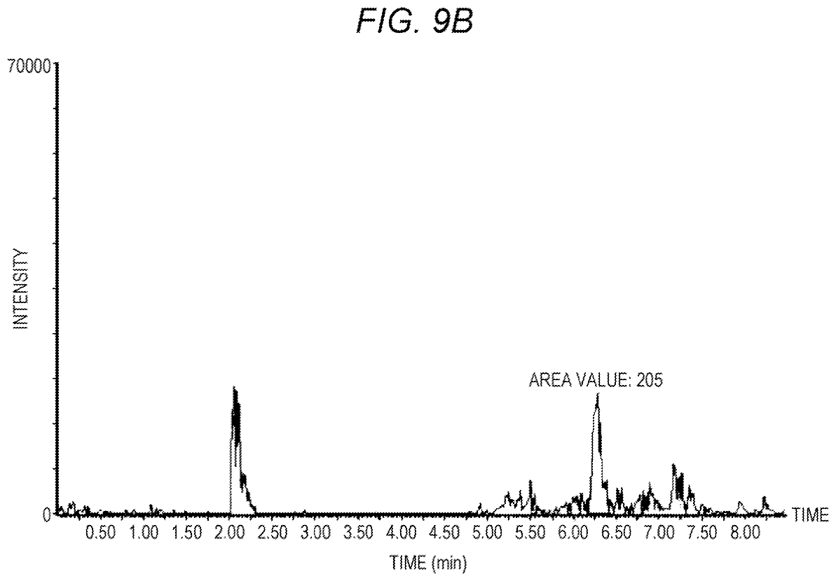

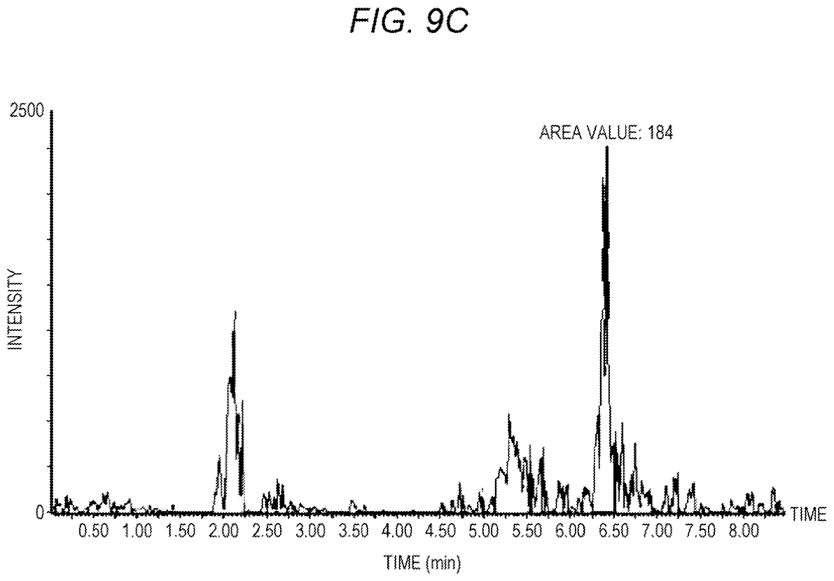

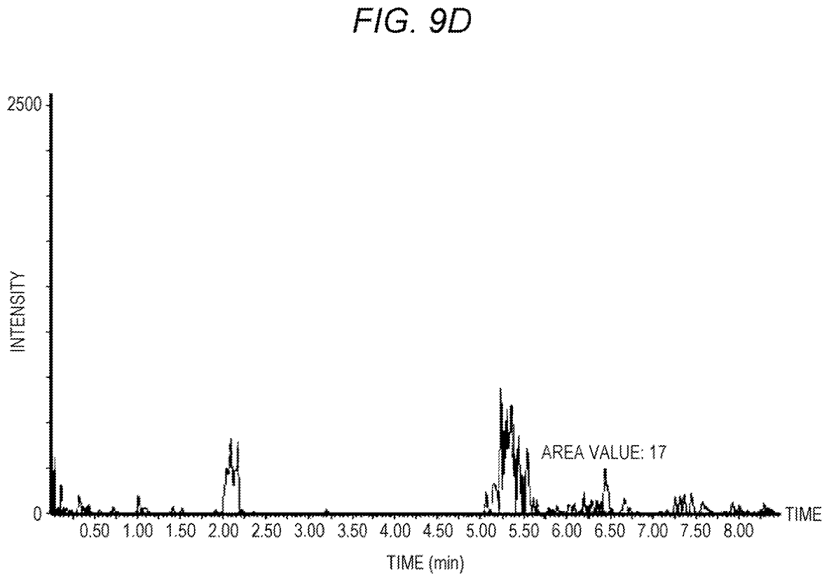

Measurement results of solution A and solution B are shown in FIGS. 8A to 9D, respectively. FIG. 8A shows a measurement result of solution X using 190 pg/ml Aβ40 peptide, FIG. 8B shows a measurement result of solution Y using 190 pg/ml Aβ40 peptide, FIG. 8C shows a measurement result of solution X using 103 pg/ml Aβ42 peptide, FIG. 8D shows a measurement result of solution Y using 103 pg/ml Aβ42 peptide, FIG. 9A shows a measurement result of solution X using 50 pg/ml Aβ40 peptide, FIG. 9B shows a measurement result of solution Y using 50 pg/ml Aβ40 peptide, FIG. 9C shows a measurement result of solution X 26 pg/ml Aβ42 peptide, and FIG. 9D show a measurement result of solution Y using 26 pg/ml Aβ42 peptide. From these results, in the examples of Aβ peptides at all concentrations, the area value was larger when using the solution X than when using the solution Y. It was suggested that loss of Aβ peptide occurred when the process as described in (2.3) above was used in sample treatment.

### Example 4: Comparison of Measured Value by Immunoassay with Measured Value by Mass Spectrometry

The Aβ peptide was released from the complex using a basic solution containing an organic solvent as a releasing agent, the obtained eluate was subjected to immunoassay or mass spectrometry, respectively, and the obtained measured values were compared.

(1) Reagent Preparation

(1.1) Plasma Sample

Eighteen specimens of plasma derived from healthy subjects were purchased and used.

(1.2) Aβ Peptide

The Aβ40 peptide and Aβ42 peptide described in (1.3) of Example 1 were used.

(1.3) Peptide Spike Sample

Of the plasma samples in (1.1) above, 5 specimens were mixed to prepare a mixed sample. This mixed sample was divided into eight, and the Aβ40 peptide and Aβ42 peptide of (1.2) above were added to each thereof in an amount which an Aβ40 peptide concentration after addition was increased by 48.5 pg/mL, 57.7 pg/mL, 56.8 pg/mL, 112.0 pg/mL, 120.3 pg/mL, 127.8 pg/mL, 248.0 pg/mL or 513.7 pg/mL, and in an amount which an Aβ42 peptide concentration after addition was increased by 5.7 pg/mL, 6.6 pg/mL, 7.5 pg/mL, 13.4 pg/mL, 13.7 pg/mL, 14.7 pg/mL, 29.3 pg/mL or 54.2 pg/mL to prepare peptide spike samples.

(1.4) Antibody that Specifically Binds to Aβ Peptide

The 6E10 antibody described in (1.2) of Example 1 was used. As the internal standard substance, 15N-Aβ40 and 15N-Aβ42 described in (1.2) of Example 1 were used. 15N-Aβ40 and 15N-Aβ42 were prepared by suspending them in PBS solutions containing 3% BSA so as to be 500 pg/ml, respectively.

(1.5) Sample for Preparing Calibration Curve in Mass Spectrometry

The Aβ40 peptide was suspended in PBS solutions containing 3% BSA, so as to be 10.8 pg/ml, 21.7 pg/ml, 43.3 pg/ml, 86.6 pg/ml, 173.2 pg/ml, 346.4 pg/ml and 692.8 pg/ml, respectively. The Aβ42 peptide was suspended in PBS solutions containing 3% BSA, so as to be 2.8 pg/ml, 5.6 pg/ml, 11.3 pg/ml, 22.6 pg/ml, 45.2 pg/ml, 90.3 pg/ml and 180.6 pg/mL, respectively, to prepare as samples for preparing a calibration curve.

(1.6) Calibrator for HISCL (Registered Trademark)-5000 Measurement

As calibrators for HISCL (registered trademark)-5000 measurement, the Aβ40 peptide was suspended in a solution at pH 7.0 containing 0.1% BSA, 0.14 M triethanolamine, 0.15 M NaCl and 0.1% NaN₃ so as to be 0 pg/ml, 8.6 pg/ml, 33.3 pg/ml, 99.2 pg/ml, 319.1 pg/ml and 1188.1 pg/ml, respectively.

(1.7) Preparation of Basic Solution Containing Organic Solvent

As a basic solution (releasing agent) containing an organic solvent, a 1.68% ammonia and 30% acetonitrile solution was prepared as described in (1.4) of Example 1.

(2) Measurement Using Mass Spectrometry

(2.1) Immunoprecipitation

A 250 μl of the plasma sample of (1.1) above, the peptide spike sample of (1.3) above, or each of the Aβ40 peptide solution or Aβ42 peptide solution prepared in (1.5) above was added to a 1.5 ml sample tube (Eppendorf AG). To each sample tube containing the above solution was added 250 μl of the solution containing 15N-Aβ40 and 15N-Aβ42 prepared in (1.4) above, and the mixture was allowed to stand at room temperature for 30 minutes. After standing the sample tube, 40 μl of the suspension of magnetic particles (4 mg antibody/0.4 mg magnetic particles) immobilized with 6E10 antibody prepared in (1.4) above was added to each sample solution, and the mixture was inverted and mixed for 1 hour using a rotator at room temperature. These solutions were focused using a magnetic stand to remove supernatant.

(2.2) Washing

After removing the supernatant, 1 mL of a PBS solution containing 3% BSA was added to the magnetic particles remaining in the sample tube, mixed, and then magnetized again to remove supernatant. This operation was performed twice with 1 mL of the PBS solution containing 3% BSA, twice with 1 mL of a 50 mM ammonium acetate solution and once with 1 mL of ultrapure water successively to wash the magnetic particles.

(2.3) Release of Aβ Peptide

After washing the magnetic particles in (2.2) above, 25 μL of the releasing agent prepared in (1.7) above was added to the remaining magnetic particles after removing the washing liquid, mixed, and allowed to stand for 1 minute. The magnetic particles were magnetically collected again, and supernatant was recovered as an eluate.

(2.4) Mass Spectrometry

The eluate prepared in (2.3) above was subjected to LC-MS/MS for MRM measurement. Conditions for LC-MS/MS were the same as in (2) of Example 1.

(3) Measurement Using Immunoassay

Separately from (2) above, Aβ peptide concentration was measured for the plasma sample of (1.1) above and the peptide spike sample of (1.3) above by an immunoassay using HISCL (registered trademark)-5000. An R1 reagent (capture antibody reagent) was prepared by labeling 82E1 antibody with biotin by a conventional method and dissolving it in a buffer at pH 7.5 containing 1% BSA, 0.1 M Tris-HCl, 0.15 M NaCl and 0.1% NaN₃. As an R2 reagent (solid phase), a HISCL (registered trademark) R2 reagent (Sysmex Corporation) containing streptavidin-bound magnetic particles was used. An R3 reagent (detection antibody reagent) was prepared by labeling 1A10 antibody with alkaline phosphatase (ALP) by a conventional method and dissolving it in a buffer at pH 7.5 containing 1% BSA, 0.1 M Tris-HCl, 0.15 M NaCl and 0.1% NaN₃. As an R4 reagent (measurement buffer solution), a HISCL R4 reagent (Sysmex Corporation) was used. As an R5 reagent (ALP substrate solution), a HISCL R5 reagent (Sysmex Corporation) was used.

Measurement procedure using HISCL (registered trademark)-5000 was carried out in the same manner as in (2) of Example 2 except that the plasma sample of (1.1) above or the peptide spike sample of (1.3) above was used.

(4) Calculation of Correlation Coefficient

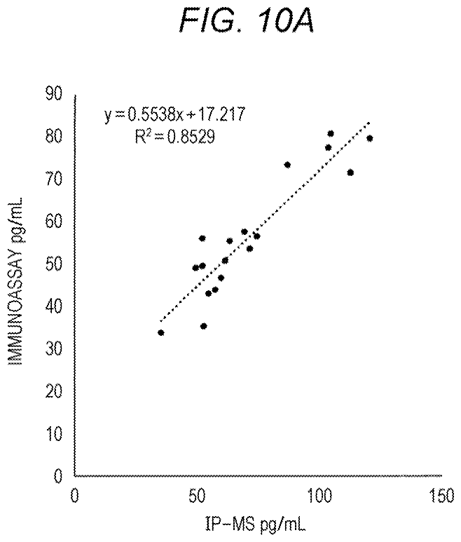

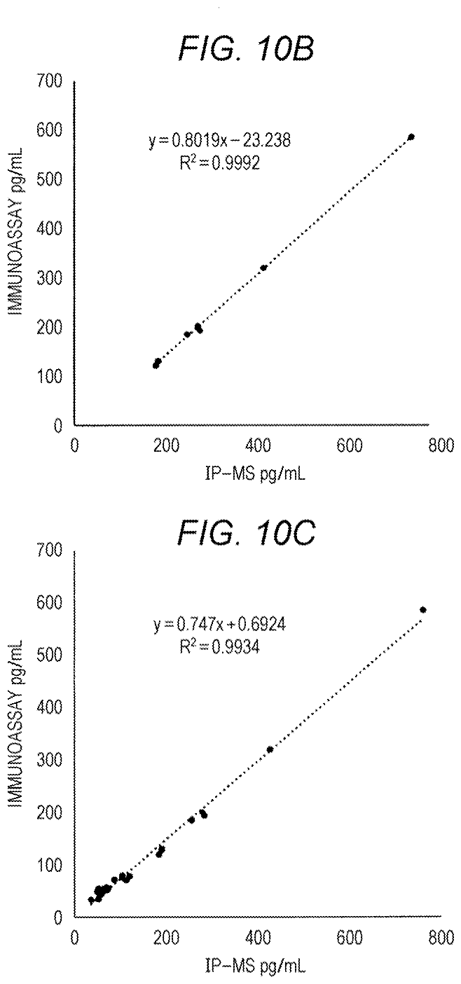

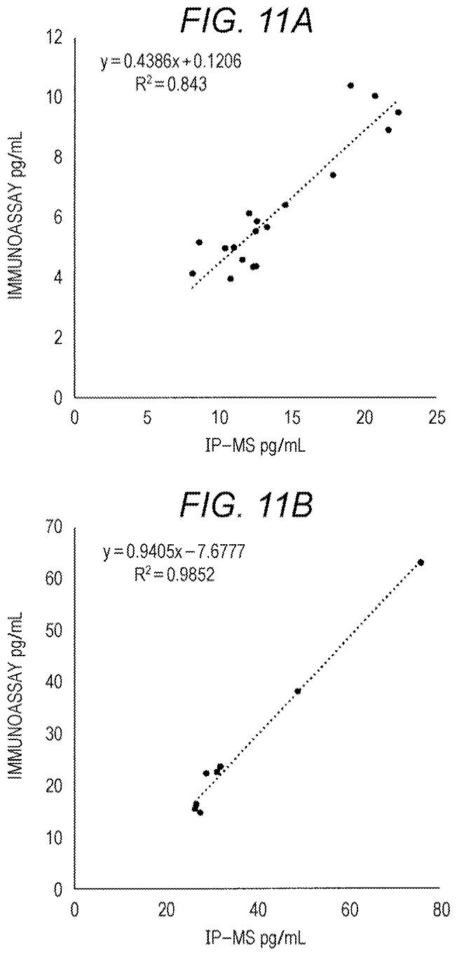

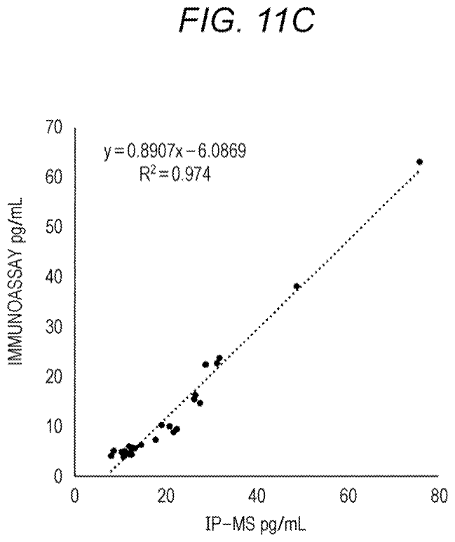

As to the measurement results of Aβ40 peptide and Aβ42 peptide measured in (2) and (3) above, results of measurement using mass spectrometry and results of measurement using HISCL (registered trademark)-5000 were plotted on a horizontal axis and a vertical axis, respectively, and results of calculating correlation coefficient r using the plotted data are shown in FIGS. 10 and 11. FIG. 10A is a graph showing the measurement results of Aβ40 peptide in the plasma sample, FIG. 10B is a graph showing the measurement results of Aβ40 peptide in the peptide spike sample, and FIG. 10C is a graph including both measurement results of FIGS. 10A and 10B. FIG. 11A is a graph showing the measurement results of Aβ42 peptide in the plasma sample, FIG. 11B is a graph showing the measurement results of Aβ42 peptide in the peptide spike sample, and FIG. 11C is a graph including both measurement results of FIGS. 11A and 11B.

(5) When Correlation Coefficient is Measured by Changing Capture Antibody

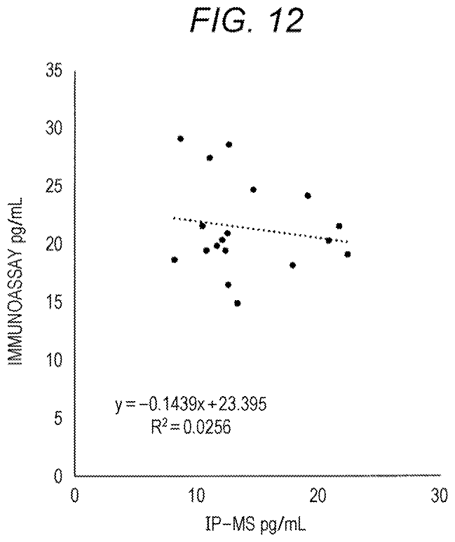

As a comparative example, in (3) above, the same experiment was carried out by replacing the capture antibody for Aβ42 peptide from 82E1 to 6E10. The 6E10 antibody is an antibody that recognizes 3rd to 8th regions counting from the N-terminal amino acid residue of the Aβ peptide as an epitope. FIG. 12 shows results of plotting in the same manner as in (4) above using the measurement results and the measurement results of Aβ42 peptide measured in (2) above. From FIG. 12, it was shown that when the capture antibody was 6E10 antibody, the result of immunoassay did not correlate with the result of mass spectrometric measurement. From the results of FIG. 12 and the results of FIGS. 10 to 11, it was shown that there was a high correlation between the measurement result using mass spectrometry and the measurement using immunoassay by using 82E1 antibody as the capture antibody.

What is claimed is:

1. A method for measuring an Aβ peptide in a blood sample in vitro, comprising
measuring the Aβ peptide by an immunoassay using an antibody set comprising a capture antibody and a detection antibody that specifically bind to the Aβ peptide,
wherein the capture antibody is an antibody that binds to an epitope comprising an N-terminal residue of the Aβ peptide, and the detection antibody is an antibody that binds to an epitope different from the epitope to which the capture antibody binds, and the Aβ peptide is at least one selected from the group consisting of Aβ40 or Aβ42.

2. The method according to claim 1, wherein
when a measured value X and a measured value Y are subjected to linier regression, a correlation coefficient r is calculated to be 0.8 or more,
wherein the measured value X is a measured value of Aβ peptide measured by HISCL (registered trademark)-5000 using the capture antibody and the detection antibody, and the measured value Y is a measured value of Aβ peptide measured by mass spectrometry,
the measurement by HISCL (registered trademark)-5000 comprises measuring the Aβ peptide in the blood sample by the capture antibody and the detection antibody labeled with alkaline phosphatase,
the measurement by mass spectrometry comprises: immunoprecipitating the Aβ peptide in the blood sample with anti-Aβ monoclonal antibody 6E10; releasing the Aβ peptide from a complex of the immunoprecipitated Aβ peptide and the antibody by a solution containing 0.56% ammonia and 40% acetonitrile; separating the solution comprising the released Aβ peptide by liquid chromatography with ACQUITY (registered trademark) UPLC (registered trademark) H-class biosystem using ACQUITY (registered trademark) UPLC (registered trademark) peptide BEH Cis column; and measuring the separated Aβ peptide using Xevo (registered trademark) TQ-XS triple quadrupole mass spectrometer using electrospray ionization, and
the measurement by the mass spectrometer is a multiple reaction monitoring (MRM) measurement, the mass spectrometer is used in positive ion measurement mode in the MRM measurement, and MRM transitions are precursor ion/product ion of 1083.4/1054 for Aβ40 peptide and precursor ion/product ion of 1129.5/1078.8 for Aβ42 peptide.

3. The method according to claim 1, wherein
when a measured value X and a measured value Y are subjected to linier regression, a correlation coefficient r is calculated to be 0.8 or more,
wherein the measured value X is a measured value of Aβ peptide measured by a fully automated immunoassay system using the capture antibody and the detection antibody, and the measured value Y is a measured value of Aβ peptide measured by mass spectrometry,
the measurement by the fully automated immunoassay system comprises measuring the Aβ peptide in the blood sample using the capture antibody and the detection antibody labeled with alkaline phosphatase, and
the measurement by mass spectrometry comprises: immunoprecipitating the Aβ peptide in the blood sample with anti-Aβ monoclonal antibody 6E10; releasing the Aβ peptide from a complex of the immunoprecipitated Aβ peptide and the antibody by a basic solution containing an organic solvent; separating the solution containing the released Aβ peptide by liquid chromatography; and ionizing the separated Aβ peptide and measuring the separated Aβ peptide with a quadrupole mass spectrometer.

4. The method according to claim 2, wherein the correlation coefficient r is 0.85 or more.

5. The method according to claim 3, wherein the correlation coefficient r is 0.85 or more.

6. The method according to claim 2, wherein the correlation coefficient r is 0.9 or more.

7. The method according to claim 3, wherein the correlation coefficient r is 0.9 or more.

8. The method according to claim 1, wherein the detection antibody is an antibody that binds to an epitope comprising a C-terminal residue of the Aβ peptide.

9. The method according to claim 1, wherein the epitope of the capture antibody is comprised in a region consisting of 1st to 16th amino acid residues counting from an N-terminus of the Aβ peptide, and the epitope of the detection antibody is comprised in a region consisting of 35th to 40th or 36th to 42nd amino acid residues counting from the N-terminus of the Aβ peptide.

10. The method according to claim 1, wherein the capture antibody is a monoclonal antibody and the detection antibody is a monoclonal antibody.

11. The method according to claim 1, wherein pH of the basic solution is 11.4 or more.

12. The method according to claim 1, wherein the basic solution comprises an ammonium ion.

13. The method according to claim 1, wherein the capture antibody is immobilized on a solid phase.

14. The method according to claim 13, wherein the solid phase is a magnetic particle.

15. The method according to claim 1, wherein the detection antibody is labeled with a labeling substance.

16. The method according to claim 15, wherein the labeling substance is an enzyme.

17. The method according to claim 15, wherein the enzyme is at least one selected from alkaline phosphatase, peroxidase, β-galactosidase, glucosidase, polyphenol oxidase, tyrosinase, acid phosphatase, and luciferase.

18. A method for measuring an Aβ peptide in a blood sample in vitro, comprising forming on a solid phase a complex comprising a capture antibody, the Aβ peptide and a detection antibody, the detection antibody being labeled with a labeling substance, and
detecting the complex based on the labeling substance in the complex whereby the Aβ peptide is measured,
wherein the capture antibody is an antibody that binds to an epitope comprising an N-terminal residue of the Aβ peptide, and the detection antibody is an antibody that binds to an epitope different from the epitope to which the capture antibody binds, and the Aβ peptide is at least one selected from the group consisting of Aβ40 or Aβ42.

19. The method according to claim 18, wherein the capture antibody is at least one selected from the group consisting of an 82E1 antibody and a 2H4 antibody, and the detection antibody is at least one selected from the group consisting of a 1A10 antibody and an H31L21 antibody.

20. A method for measuring an Aβ peptide in a blood sample in vitro, comprising
measuring an Aβ40 by an immunoassay using: a capture antibody which is at least one selected from the group consisting of an 82E1 antibody and a 2H4 antibody; and a detection antibody which is a 1A10 antibody, and
measuring an Aβ42 by an immunoassay using: a capture antibody which is at least one selected from the group consisting of an 82E1 antibody and a 2H4 antibody; and a detection antibody which is an H31L21 antibody.


SEQUENCE LISTING (2 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]